(19) United States
(12) Patent Application Publication
(10) Pub. No.: US 2026/0232757 A1
(43) Pub. Date: Aug. 13, 2026

(54) OPHTHALMIC COMPOSITION FOR OCULAR SURGERY PREPARATION IN PATIENTS WITH OCULAR SURFACE DISEASE

(71) Applicant: NOVALIQ GMBH, Heidelberg (DE)

(72) Inventors: Sonja KRÖSSER, Heidelberg (DE); Alice MEIDES, Heidelberg (DE); Markus BEIER, Weinheim (DE)

(21) Appl. No.: 19/155,206

(22) PCT Filed: Feb. 12, 2024
(86) PCT No.: PCT/EP2024/053516

§ 371 Date: Aug. 8, 2025

Related U.S. Application Data
(60) Provisional application No. 63/484,688, filed on Feb. 13, 2023.

(51) Int. Cl. A61K 38/13; A61K 9/00; A61K 9/08; A61K 47/10; A61P 27/12; A61P 41/00

(52) U.S. Cl. CPC A61K 38/13; A61K 9/0048; A61K 9/08; A61K 47/10; A61P 27/12; A61P 41/00

(57) ABSTRACT

The present disclosure provides methods for (a) preparing a patient for ocular surgery, (b) treating ocular surface disease in a patient preparing for ocular surgery, and (c) performing biometry measurements in preparation for ocular surgery, wherein the methods comprise topically administering once to four times daily to the eye of the patient a composition comprising about 0.05-0.1% (w/v) cyclosporine dissolved in a semifluorinated alkane, optionally with an alcoholic co-solvent.

### BACKGROUND

Ocular surgery includes cataract surgery, refractive surgery (including keratomileusis, keratoplasty, radial keratotomy, and intraocular lens implantation), glaucoma surgery (including canaloplasty, laser trabeculoplasty, iridotomy, iridectomy, sclerostomy, trabeculectomy, and viscocanalostomy), laser eye surgery (e.g., laser-assisted in situ keratomileusis or LASIK, and photorefractive keratectomy, also known as LASEK, laser-assisted epithelial keratomileusis), and vitreoretinal surgery (e.g., vitrectomy, retinal detachment repair). Because the eye is a particularly fragile organ, extreme care is required both before, during, and after, any ocular surgery. It is also necessary to ensure that the patient is in optimal condition for undergoing the surgery beforehand.

A particularly important type of ocular surgery is cataract surgery, which aims to restore vision to an eye affected by cataracts.

The lens is the structure in the human eye which is responsible for focusing incoming light onto the photosensitive cells of the retina. The lens is part of the anterior segment of the eye, lying behind the iris and in front of the vitreous body. All light perceived by the eye must pass through the lens, and therefore, maintaining the transparency of the lens to light is critical to vision.

The lens functions as part of an integrated system of muscle and connection tissue which allows the shape of the lens to be adjusted in order to change the refractive index of the lens. This process is called accommodation and allows a person to adjust their visual focus from far objects to near object, and vice versa. As a person ages, however, the lens can become stiffer and less able to adjust its shape.

Cataracts are opacities of the lens. While some cataracts are small and do not require treatment, in many cases cataracts are large enough to interference with vision by blocking light from reaching the retina. Cataracts often develop as part of the aging process, but they can also be congenital and they can form in response to ocular injury, including trauma and radiation. Cataracts often develop slowly and they can affect one eye or both eyes. Symptoms of cataracts include faded colors, blurred vision, double vision, halos around light sources, bright light sensitivity, difficulty reading, and difficulty seeing at night.

Cataracts are a widespread problem, especially with the aging of the population. If left untreated, cataracts will advance to blindness. Cataracts are the cause of 51% of cases of blindness worldwide, and 33% of cases of vision impairment worldwide. In the United States, cataracts occur in about 68% of people over the age of 80.

Cataracts may be categorized as partial or complete, stationary or progressive, and hard or soft. Cataracts are also categorized by their location and pathology into three primary categories: nuclear sclerotic, cortical, and posterior subscapular. Nuclear sclerotic cataracts affect the central region of the lens, the nucleus, beginning as a gradual hardening and yellowing of lens material. Over time, the effected zone expands to the other layers of the lens. Cortical cataracts begin in the outer layer of the lens, the cortex, and gradually extend from the periphery to the center of the lens. Posterior subscapular cataracts begin on the posterior surface of the lens beneath the lens capsule. While nuclear and cortical cataracts develop slowly over the course of years, subscapular cataracts can progress rapidly, over just a few months.

Cataracts may be classified according to the LOCS III system (Lens Opacities Classification System III; see Chylack et al., Arch. Ophthalmol. 111(6):831-36 (1993)). The system provides a score ranging from trace to Grade 4+, using different criteria for the different types of cataracts. For nuclear sclerotic cataracts, the degree of nuclear coloration (yellowing) and opalescence is evaluated. For cortical cataracts, the extent of cortical spoking is evaluated (percentage of lens showing spoking). For posterior subscapular cataracts, the extent of opacification is evaluated (percentage of the capsule showing opacity).

A lens with a cataract cannot be repaired, so the only effective treatment for advanced cataracts is surgical removal of the damaged lens and replacement by an artificial intraocular lens, typically made of a polymer such as polymethylmethacrylate or silicone. Cataract surgery has become quite common, with about six million lenses implanted annually worldwide. The surgical procedure is normally performed on an outpatient basis with only local (ocular) anesthesia. An experienced ophthalmologist can perform a cataract surgery in less than 30 minutes.

The most common reason for cataract surgery is age-related cataracts, primarily of the nuclear sclerotic type. Typically, surgery is warranted when the cataract becomes grade 3+ or 4+. Once surgery is warranted, it should be carried out as soon as possible. If surgery becomes delayed, the lens will continue to become increasingly white and dense, and the surgery becomes more difficult to perform with higher risks of complications for the patient.

One of the major risk factors for cataract surgery (as well as other ocular surgeries, such as refractive surgery), is undertreated or untreated ocular surface disease, such as corneal surface damage and tear film deficiency or disruption. Indeed, studies have shown that up to 50% of patients presenting for ocular surgery are unexpectedly found to be suffering from such pre-existing conditions, which must be quickly treated in order to permit the patient to undergo the ocular surgery that is necessary for saving or restoring their vision.

In some studies, it has been found that pre-existing undertreated or untreated ocular surface disease, in particular corneal surface damage and tear film deficiency or disruption, are leading causes of post-surgical discomfort, complications, or patient dissatisfaction. The traumatic nature of the ocular surgery may exacerbate such pre-existing conditions even when the patient was not consciously aware of the condition beforehand. Numerous undiagnosed systemic diseases may contribute to ocular surface disease, including collagen vascular disease, rheumatoid arthritis, Graves disease and other thyroid disorders, Sjogren syndrome, vitamin A deficiency, and diabetes mellitus. Some very common drugs may also contribute to these pre-existing conditions, including antihistamines, tricyclic antidepressants, selective serotonin reuptake inhibitors, diuretics, and beta-blockers. Some ocular diseases which may also lead to such pre-existing conditions include chronic conjunctivitis (such as allergic conjunctivitis, infectious conjunctivitis or tobacco-smoke induced conjunctivitis), blepharitis, dry eye disease, Meibomian gland dysfunction, xerophthalmia, and alacrima, as well as contact lens use or overuse.

Pre-operative evaluation for many ocular surgeries, especially cataract surgery and refractive surgery, requires an accurate anatomical assessment of the eye (biometry measurement), including determination of the axial length (AL), keratometry (cornea curvature) and anterior chamber depth (ACD). Biometry is an essential step before cataract surgery and refractive intraocular lens implantation, as selection of the wrong lens size, shape or curvature, can significantly reduce the chance for a successful outcome. Yet, corneal surface abnormalities can lead to inaccurate keratometry assessments which are essential for properly calculating the power requirements for an intraocular lens. Therefore, ensuring a maximally healthy cornea before biometry measurement are taken is essential in maximizing positive outcomes in ocular surgery.

There is no effective treatment to improve the ocular surface or corneal surface with an early onset of action. Current treatment options have rather weak effects on the ocular surface and the time to onset of effect takes long (e.g., about 4-6 months for Restasis®, and about 3 months for Ikervis®). Corticosteroids may act faster, however, evidence of their effect on improving the ocular surface in randomized controlled trials is limited. Moreover, current treatment options have additional drawbacks, including instillation site irritation due to the surfactants and preservatives in the formulation. At present, the most common method of treating ocular surface disease prior to cataract surgery, or prior to biometry measurements for cataract surgery, is using a slow-acting dry eye disease medication (e.g., Restasis®), sometimes in combination with an ophthalmic corticosteroid. However, as noted above, it takes several months of treatment to be effective.

Accordingly, there is a need for an improved means and method for pre-operatively treating patients in preparation for ocular surgery to reduce ocular surface disease, in particular corneal surface damage. In particular, there is a need for an improved means and methods for pre-operatively improving ocular surface disease, in particular corneal surface damage, prior to biometry measurements in subjects suffering from ocular surface disease, such as in subjects suffering from corneal surface damage. There is especially a need to do so rapidly with minimal side effects (e.g., irritation) in order to permit the patient to proceed quickly to the ocular surgery after most accurate biometry measurements. It is thus an object of the present invention to provide compositions and methods which are effective in meeting these needs. Further objects of the invention will be clear on the basis of the following description of the invention, examples and claims.

### BRIEF SUMMARY

In a first aspect, the present disclosure provides a method for preparing a patient for ocular surgery (e.g., cataract surgery), the method comprising topically administering once to four times daily to the eye of the patient a composition comprising about 0.05-0.1% (w/v) cyclosporine dissolved in a semifluorinated alkane, optionally with an alcoholic co-solvent.

In a second aspect, the present disclosure provides a method for treating ocular surface disease (e.g., corneal surface damage, tear film deficiency or tear film disruption), in a patient preparing for ocular surgery (e.g., cataract surgery), the method comprising topically administering once to four times daily to the eye of the patient a composition comprising about 0.05-0.1% (w/v) cyclosporine dissolved in a semifluorinated alkane, optionally with an alcoholic co-solvent.

In a third aspect, the present disclosure provides a method for performing biometry measurements for ocular surgery (e.g., cataract surgery) comprising topically administering once to four times daily to the eye of the patient a composition comprising about 0.05-0.1% (w/v) cyclosporine dissolved in a semifluorinated alkane, optionally with an alcoholic co-solvent, prior to the biometry measurements.

In particular embodiments of the first, second, and third aspects, the methods of the present disclosure achieve their goals with a rapid onset of effect (e.g., less than 2 weeks).

### DETAILED DESCRIPTION

In a first aspect, the present disclosure provides a method (Method 1) for preparing a patient for ocular surgery (e.g., cataract surgery), the method comprising topically administering once to four times daily (e.g., two times daily) to the eye of the patient (e.g., the eye undergoing ocular surgery) a composition comprising about 0.05-0.1% (w/v) cyclosporine dissolved in a semifluorinated alkane (e.g., 1-perfluorobutylpentane), optionally with an alcoholic co-solvent (e.g., ethanol).

In further embodiments of the first aspect, the present disclosure provides:
1.1 Method 1, wherein the ocular surgery is selected from cataract surgery, refractive surgery (including keratomileusis, keratoplasty, radial keratotomy, and intraocular lens implantation), glaucoma surgery (including canaloplasty, laser trabeculoplasty, iridotomy, iridectomy, sclerostomy, trabeculectomy, and viscocanalostomy), laser eye surgery (e.g., laser-assisted in situ keratomileusis or LASIK, and photorefractive keratectomy, also known as LASEK, laser-assisted epithelial keratomileusis), and vitreoretinal surgery (e.g., vitrectomy, retinal detachment repair);
1.2 Method 1.1, wherein the ocular surgery is cataract surgery or refractive surgery;
1.3 Method 1.2, wherein the ocular surgery is cataract surgery or refractive surgery via intraocular lens implantation;
1.4 Method 1, or any of 1.1-1.3, wherein the method further comprises the step of determining the degree of ocular surface disease (e.g., corneal surface damage, tear film deficiency, and/or tear film disruption) in preparation for the ocular surgery;
1.5 Method 1.4, wherein the determination of the degree of ocular surface disease comprises the step of determining the degree of corneal surface damage and/or tear film deficiency or disruption;
1.6 Method 1.5, wherein the degree of corneal surface damage is determined by the total corneal fluorescein staining score and/or the central corneal fluorescein staining score, and/or wherein the degree of tear film deficiency or disruption is determined by the tear film breakup time (TFBUT) or Schirmer's test;
1.7 Method 1, or any of 1.1-1.6, wherein the method further comprises the step of performing biometry measurements in preparation for the ocular surgery;
1.8 Method 1.7, wherein the biometry measurements comprise keratometry and/or optical biometry, such as, measurement of corneal curvature (e.g., anterior corneal curvature, posterior corneal curvature), axial length, anterior chamber depth, lens thickness, and/or corneal power, of the eye undergoing ocular surgery;
1.9 Method 1, or any of 1.1-1.8, wherein the semifluorinated alkane is 1-perfluorobutylpentane;
1.10 Method 1, or any of 1.1-1.9, wherein the composition comprises 1-perfluorobutylpentane and 2-perfluorobutylpentane;
1.11 Any of Methods 1.9-1.10, wherein the composition comprises 1-perfluorobutylpentane and 2-perfluorobutylpentane, in a weight ratio of about 95:5, or about 96:4, or about 97:3, or about 98:2, or about 99:1, or about 99.5:0.5, or about 99.6:0.4, or about 99.7:0.3, or about 99.8:0.2, or about 99.9:0.1;
1.12 Any of Methods 1.9-1.11, wherein the composition comprises up to 5% (w/w) of 2-perfluorobutylpentane, or up to 3% (w/w), or up to 2% (w/w), or up to 1% (w/w), or up to 0.5% (w/w), or up to 0.4% (w/w), or up to 0.3% (w/w), or up to 0.2% (w/w), or up to 0.1% (w/w) of 2-perfluorobutylpentane, optionally wherein the composition comprises at least 0.01% (w/w) or at least 0.1% (w/w) or at least 0.3% (w/w) of 2-perfluorobutylpentane;
1.13 Method 1, or any of 1.1-1.12, wherein the composition is free or substantially free of water, e.g., less than 2 wt. %, or less than 1 wt. %, or less than 0.5 wt. %, or less than 0.2 wt. %, or less than 0.1 wt. % water by weight of the composition;
1.14 Method 1, or any of 1.1-1.13, wherein the composition is free or substantially free of any preservatives, e.g., less than 3 wt. %, or less than 2 wt. %, or less than 1 wt. %, or less than 0.5 wt. %, or less than 0.1 wt. % preservatives by weight of the composition, or wherein the composition is not formulated to include any preservatives;
1.15 Method 1, or any of 1.1-1.14, wherein the composition further comprises an alcoholic co-solvent;
1.16 Method 1.15, wherein the alcoholic co-solvent is ethanol;
1.17 Any of Methods 1.15-1.16, wherein the composition comprises the alcoholic co-solvent in an amount of 0.01 to 5% (w/w), or 0.05 to 4% (w/w), or 0.1 to 3% (w/w), or 0.2 to 2% (w/w), or 0.2 to 1.5% (w/w), or 0.2 to 1.0% (w/w), or 0.5 to 1.2% (w/w), or about 0.5% (w/w), or about 1.0% (w/w);
1.18 Method 1, or any of 1.1-1.17, wherein the composition comprises about 0.05-0.1% (w/v) cyclosporine dissolved in 1-perfluorobutylpentane;
1.19 Method 1.18, wherein the composition comprises about 0.1% (w/v) cyclosporine dissolved in 1-perfluorobutylpentane;
1.20 Method 1.19, wherein the composition comprises or essentially consists of about 0.1% (w/v) cyclosporine dissolved in 1-perfluorobutylpentane with about 1% (w/w) ethanol;
1.21 Method 1.20, wherein the composition comprises or essentially consists of about 0.1% (w/v) cyclosporine dissolved in 1-perfluorobutylpentane with about 1% (w/w) ethanol and up to about 1% (w/w) 2-perfluorobutylpentane;
1.22 Method 1.21, wherein the composition consists of about 0.1% (w/v) cyclosporine dissolved in 1-perfluorobutylpentane with about 1% (w/w) ethanol and up to about 0.5% (w/w) 2-perfluorobutylpentane;
1.23 Method 1, or any of 1.1-1.22, wherein the composition is administered four times daily;
1.24 Method 1, or any of 1.1-1.22, wherein the composition is administered three times daily;
1.25 Method 1, or any of 1.1-1.22, wherein the composition is administered two times daily;
1.26 Method 1, or any of 1.1-1.22, wherein the composition is administered once daily;
1.27 Method 1, or any of 1.1-1.26, wherein the amount of cyclosporine administered in a single dose per eye is about 8-12 μg, e.g., 9-11 μg, 9-10 μg, or about 9 μg, or about 10 μg cyclosporine;
1.28 Method 1, or any of 1.1-1.27, wherein the volume administered in a single dose per eye is about 8-12 μL, e.g., 9-11 μL, e.g., 9-10 μL, or about 9 μl or about 10 μL;
1.29 Method 1, or any of 1.1-1.28, wherein the patient is administered the composition for at least a period of 7 days, or for at least 14 days, or for at least 21 days, or for at least 28 days, or for at least 35 days; or for at least 42 days;
1.30 Method 1, or any of 1.1-1.29, wherein the step of determining the degree of ocular surface disease (e.g., corneal surface damage, tear film efficiency and/or tear film disruption) precedes the step of performing biometry measurements;
1.31 Method 1, or any of 1.1-1.30, wherein the last day of administration of the composition precedes the patient's biometry measurements, e.g., by at least 1 day, or at least 2 days, or at least 3 days, or at least 5 days, or at least 7 days, or 1-7 days;
1.32 Method 1, or any of 1.1-1.31, wherein the step of determining the degree of ocular surface disease (e.g., corneal surface damage, tear film deficiency and/or tear film disruption) precedes the administration of the composition;
1.33 Method 1, or any of 1.1-1.32, wherein the administration of the composition continues after the biometry measurement, optionally until 1-3 days before the patient's ocular surgery;
1.34 Method 1, or any of 1.1-1.33, wherein the administration of the composition continues after the ocular surgery;
1.35 Method 1, or any of 1.1-1.34, wherein the method further comprises the topical ophthalmic administration of a corticosteroid;
1.36 Method 1, or any of 1.1-1.34, wherein the method does not further comprise the topical ophthalmic administration of a corticosteroid;
1.37 Method 1, or any of 1.1-1.36, wherein the method does not further comprise the topical ophthalmic administration of a drug that blocks LFA-1 binding to ICAM-1 (e.g., lifitegrast);
1.38 Method 1, or any of 1.1-1.37, wherein the composition is the only topical ophthalmic composition administered to the patient during the period when the method is carried out;
1.39 Method 1, or any of 1.1-1.38, wherein the patient has not been previously diagnosed with dry eye disease prior to evaluation for the ocular surgery;
1.40 Method 1, or any of 1.1-1.38, wherein the patient has been previously diagnosed with dry eye disease prior to evaluation for the ocular surgery;
1.41 Method 1, or any of 1.1-1.40, wherein the patient has ocular surface disease (e.g., corneal surface damage, tear film deficiency, and/or tear film disruption);
1.42 Method 1, or any of 1.1-1.41, wherein the patient is first identified as having ocular surface disease (e.g., corneal surface damage, tear film deficiency, and/or tear film disruption), in evaluation for or in preparation for the ocular surgery;
1.43 Method 1, or any of 1.1-1.41, wherein the method further comprises the step of measuring or evaluating the degree of ocular surface disease, prior to administering the composition;
1.44 Method 1, or any of 1.1-1.43, wherein the method comprises the step of determining the degree of ocular surface disease (e.g., corneal surface damage, tear film deficiency, and/or tear film disruption), after the completion of the administering of the composition (e.g., after administration of the composition for at least a period of 7 days, or for at least 14 days, or for at least 21 days, or for at least 28 days or for at least 35 days or for at least 42 days), e.g., by total corneal fluorescein staining score and/or central corneal fluorescein staining score, such as for the purpose of comparison to the pre-treatment condition;
1.45 Method 1.44, wherein the method further comprises the step of comparing the degree of ocular surface disease (e.g., corneal surface damage, tear film deficiency, and/or tear film disruption) before and after the administration of the composition (e.g., before versus after administration of the composition for at least a period of 7 days, or for at least 14 days, or for at least 21 days, or for at least 28 days or for at least 35 days or for at least 42 days) to determine whether the patient can proceed with the biometry measurements and/or the ocular surgery;
1.46 Method 1, or any of 1.1-1.45, wherein the method further comprises the step of subjecting the patient to ocular surgery upon having a determination made that the ocular surface disease (e.g., corneal surface damage, tear film deficiency, and/or tear film disruption) has been reduced to a level safe and suitable for taking biometry measurements and/or performing the ocular surgery;
1.47 Method 1, or any of 1.1-1.46, wherein, before the beginning of the administration of the composition according to the method, the patient is characterized by the eye intended for ocular surgery having:
   (a) a total corneal fluorescein staining score of greater than or equal to 7 (e.g., greater than 8, 9, 10, 11, or 12), or greater than or equal to 10 (e.g., greater than 10, 11, 12 or 13), such as 7-15, 7-13, 10-15 or 10-13; and/or
   (b) a central corneal fluorescein staining score of equal or greater than 1 (e.g., greater than 1, greater than 2), such as 1-2 or 1-3 or 2-3;
1.48 Method 1, or any of 1.1-1.47, wherein after the period of treatment with the composition (e.g., after administration of the composition for at least a period of 7 days, or for at least 14 days, or for at least 21 days, or for at least 28 days or for at least 35 days or for at least 42 days) according to the method, the patient is characterized by the eye intended for ocular surgery having:
   (a) a total corneal fluorescein staining score of less than 7 (e.g., less than or equal to 1, 2, 3, 4, 5, or 6) or less than 10 (e.g., less than or equal to 3, 4, 5, 6, 7, 8 or 9); and/or a total corneal fluorescein staining score that has decreased by at least 3 grades from the pre-treatment score; and/or
   (b) a central corneal fluorescein staining score of less than or equal to 1; and/or a central corneal fluorescein staining score that has decreased by at least 1 grade from the pre-treatment score;
1.49 Method 1, or any of 1.1-1.48, wherein the method improves the patient's total corneal fluorescein staining score in the eye intended for ocular surgery by at least 3 grades before the biometry measurements are performed and/or before the ocular surgery is performed;
1.50 Method 1, or any of 1.1-1.49, wherein the method improves the patient's central corneal fluorescein staining score in the eye intended for ocular surgery by at least 1 grade before the biometry measurements are performed and/or before the ocular surgery is performed;
1.51 Method 1, or any of 1.1-1.50, wherein the method further comprises the step of subjecting the patient to biometry measurements upon reduction of the patient's total corneal fluorescein staining score in the eye intended for ocular surgery by at least 3 grades;
1.52 Method 1, or any of 1.1-1.51, wherein the method further comprises the step of subjecting the patient to ocular surgery upon reduction of the patient's total corneal fluorescein staining score in the eye intended for ocular surgery by at least 3 grades;
1.53 Method 1, or any of 1.1-1.52, wherein the method improves the patient's tear quality in the eye intended for surgery, e.g., as shown by an improved tear film break-up time (TFBUT) and/or an improved Schirmer's Test score;
1.54 Any of Methods 1.48-1.53, wherein the patient achieves the stated level of improvement after less than 8 weeks of treatment, e.g., after 1 to 6 weeks of administration of the composition, or after 1 to 5 weeks of administration of the composition, or after 1 to 4 weeks of administration of the composition, or after 2 to 6 weeks of administration of the composition, or after 2 to 5 weeks of administration of the composition, or after 2 to 4 weeks of administration of the composition, or after 2 to 3 weeks of administration of the composition or after less than 4 weeks of administration of the composition;
1.55 Method 1, or any of 1.1-1.54, wherein the patient is subjected to biometry after the administration of the composition for sufficient time to afford a reduction at least 3 grades in the tCFS score in the eye intended for ocular surgery;
1.56 Method 1, or any of 1.1-1.55, wherein the composition is administered to the patient until the patient's total corneal fluorescein staining score in the eye intended for ocular surgery is reduced by at least 3 grades;
1.57 Method 1, or any of 1.1-1.56, wherein the patient suffers from dry eye disease at the time of evaluation or preparation for the ocular surgery (e.g., cataract surgery), optionally, wherein the patient was not diagnosed with dry eye disease prior to the time of evaluation or preparation for the ocular surgery (e.g., cataract surgery);
1.58 Method 1.57 wherein the patient suffers from ocular surface disease (e.g., corneal surface damage, tear film deficiency and/or tear film disruption) caused by the dry eye disease;
1.59 Method 1, or any of 1.1-1.57, wherein the patient suffers from ocular surface disease (e.g., corneal surface damage, tear film deficiency and/or tear film disruption) not caused by dry eye disease;
1.60 Method 1.59, wherein the ocular surface disease (e.g., corneal surface damage, tear film deficiency and/or tear film disruption) is caused by corneal abrasion, ocular chemical irritation, irritation due to contact lens wear (e.g., improper contact lens wear or sensitivity to contact lens care solutions), foreign body exposure, ultraviolet light injury, or ionizing radiation injury;
1.61 Method 1, or any of 1.1-1.60, wherein the patient does not suffer from Meibomian gland dysfunction;
1.62 Method 1, or any of 1.1-1.61, wherein the patient does not suffer from blepharitis or uveitis;
1.63 Method 1, or any of 1.1-1.62, wherein the composition does not comprise latanoprost, tafluprost, bimatoprost, travoprost, unoprostone, tacrolimus, sirolimus, everolimus, timolol, nebivolol, antibacterial agents, antifungal agents, antiviral agents.

In a second aspect, the present disclosure provides a method (Method 2) for treating ocular surface disease (e.g., corneal surface damage, tear film deficiency or tear film disruption), in a patient preparing for ocular surgery (e.g., cataract surgery), the method comprising topically administering once to four times (e.g., two times) daily to the eye of the patient a composition comprising about 0.05-0.1% (w/v) cyclosporine dissolved in a semifluorinated alkane (e.g., 1-perfluorobutylpentane), optionally with an alcoholic co-solvent (e.g., ethanol).

In further embodiments of the second aspect, the present disclosure provides:
2.1. Method 2, wherein the ocular surgery is selected from cataract surgery, refractive surgery (including keratomileusis, keratoplasty, radial keratotomy, and intraocular lens implantation), glaucoma surgery (including canaloplasty, laser trabeculoplasty, iridotomy, iridectomy, sclerostomy, trabeculectomy, and viscocanalostomy), laser eye surgery (e.g., laser-assisted in situ keratomileusis or LASIK, and photorefractive keratectomy, also known as LASEK, laser-assisted epithelial keratomileusis), and vitreoretinal surgery (e.g., vitrectomy, retinal detachment repair);
2.2. Method 2.1, wherein the ocular surgery is cataract surgery or refractive surgery;
2.3. Method 2.2, wherein the ocular surgery is cataract surgery or refractive surgery via intraocular lens implantation;
2.4. Method 2, or any of 2.1-2.3, wherein the method further comprises the step of determining the degree of ocular surface disease (e.g., corneal surface damage, tear film deficiency, and/or tear film disruption) in preparation for the ocular surgery;
2.5. Method 2.4, wherein the determination of the degree of ocular surface disease comprises the step of determining the degree of corneal surface damage and/or the tear film deficiency or disruption;
2.6. Method 2.5, wherein the degree of corneal surface damage is determined by the total corneal fluorescein staining score and/or the central corneal fluorescein staining score, and/or wherein the degree of tear film deficiency or disruption is determined by the tear film breakup time (TFBUT) or Schirmer's test;
2.7. Method 2, or any of 2.1-2.6, wherein the method further comprises the step of performing biometry measurements in preparation for the ocular surgery;
2.8. Method 2.7, wherein the biometry measurements comprise keratometry and/or optical biometry, such as, measurement of corneal curvature (e.g., anterior corneal curvature, posterior corneal curvature), axial length, anterior chamber depth, lens thickness, and/or corneal power, of the eye undergoing ocular surgery;
2.9. Method 2, or any of 2.1-2.8, wherein the semifluorinated alkane is 1-perfluorobutyl-pentane;
2.10. Method 2, or any of 2.1-2.9, wherein the composition comprises 1-perfluorobutylpentane and 2-perfluorobutylpentane;
2.11. Any of Methods 2.9-2.10, wherein the composition comprises 1-perfluorobutylpentane and 2-perfluorobutylpentane, in a weight ratio of about 95:5, or about 96:4, or about 97:3, or about 98:2, or about 99:1, or about 95.5:0.5, or about 99.6:0.4, or about 99.7:0.3, or about 99.8:0.2, or about 99.9:0.1;
2.12. Any of Methods 2.9-2.11, wherein the composition comprises up to 5% (w/w) of 2-perfluorobutylpentane, or up to 3% (w/w), or up to 2% (w/w), or up to 1% (w/w), or up to 0.5% (w/w) or up to 0.4% (w/w), or up to 0.3% (w/w), or up to 0.2% (w/w), or up to 0.1% (w/w) of 2-perfluorobutylpentane, optionally wherein the composition comprises at least 0.01% (w/w) or at least 0.1% (w/w) or at least 0.3% (w/w) of 2-perfluorobutylpentanethe;
2.13. Method 2, or any of 2.1-2.12, wherein the composition is free or substantially free of water, e.g., less than 2 wt. %, or less than 1 wt. %, or less than 0.5 wt. %, or less than 0.2 wt. %, or less than 0.1 wt. % preservatives by weight of the composition;
2.14. Method 2, or any of 2.1-2.13, wherein the composition is free or substantially free of any preservatives, e.g., less than 3 wt. %, or less than 2 wt. %, or less than 1 wt. %, or less than 0.5 wt. %, or less than 0.1 wt. % water by weight of the composition, or wherein the composition is not formulated to include any preservatives;
2.15. Method 2, or any of 2.1-2.14, wherein the composition further comprises an alcoholic co-solvent;
2.16. Method 2.15, wherein the alcoholic co-solvent is ethanol;
2.17. Any of Methods 2.15-2.16, wherein the composition comprises the alcoholic co-solvent in an amount of 0.01 to 5% (w/w), or 0.05 to 4% (w/w), or 0.1 to 3% (w/w), or 0.2 to 2% (w/w), or 0.2 to 1.5% (w/w), or 0.2 to 1.0% (w/w), or 0.5 to 1.2% (w/w), or about 0.5% (w/w), or about 1.0% (w/w);
2.18. Method 2, or any of 2.1-2.17, wherein the composition comprises about 0.05-0.1% (w/v) cyclosporine dissolved in 1-perfluorobutylpentane;
2.19. Method 2.18, wherein the composition comprises about 0.1% (w/v) cyclosporine dissolved in 1-perfluorobutylpentane;
2.20. Method 2.19, wherein the composition comprises or essentially consists of about 0.1% (w/v) cyclosporine dissolved in 1-perfluorobutylpentane with about 1% (w/w) ethanol;
2.21. Method 2.20, wherein the composition comprises or essentially consists of about 0.1% (w/v) cyclosporine dissolved in 1-perfluorobutylpentane with about 1% (w/w) ethanol and up to about 2% (w/w), or up to about 1% (w/w) 2-perfluorobutylpentane;
2.22. Method 2.21, wherein the composition consists of about 0.1% (w/v) cyclosporine dissolved in 1-perfluorobutylpentane with about 1% (w/w) ethanol and up to about 2% (w/w), or up to about 0.5% (w/w) 2-perfluorobutylpentane;
2.23. Method 2, or any of 2.1-2.22, wherein the composition administered four times daily;
2.24. Method 2, or any of 2.1-2.22, wherein the composition administered three times daily;
2.25. Method 2, or any of 2.1-2.22, wherein the composition administered two times daily;
2.26. Method 2, or any of 2.1-2.22, wherein the composition administered once daily;
2.27. Method 2, or any of 2.1-2.26, wherein the amount of cyclosporine administered in a single dose per eye is about 8-12 μg, e.g., 9-11 μg, 9-10 μg, or about 9 μg, or about 10 μg cyclosporine;
2.28. Method 2, or any of 2.1-2.27, wherein the volume administered in a single dose per eye is about 8-12 μL, e.g., 9-11 μL, e.g., 9-10 μL, or about 9 μl, or about 10 μL;
2.29. Method 2, or any of 2.1-2.28, wherein the patient is administered the composition for at least a period of 7 days, or for at least 14 days, or for at least 21 days, or for at least 28 days, or for at least 35 days; or for at least 42 days;
2.30. Method 2, or any of 2.1-2.29, wherein the step of determining the degree of ocular surface disease (e.g., corneal surface damage, tear film deficiency and/or tear film disruption) precedes the step of performing biometry measurements;
2.31. Method 2, or any of 2.1-2.30, wherein the last day of administration of the composition precedes the patient's biometry measurements, e.g., by at least 1 day, or at least 2 days, or at least 3 days, or at least 5 days, or at least 7 days, or 1-7 days;
2.32. Method 1, or any of 2.1-2.31, wherein the step of determining the degree of ocular surface disease (e.g., corneal surface damage, tear film deficiency and/or tear film disruption) precedes the administration of the composition;
2.33. Method 2, or any of 2.1-2.32, wherein the administration of the composition continues after the biometry measurement, optionally until 1-3 days before the patient's ocular surgery;
2.34. Method 2, or any of 2.1-2.33, wherein the administration of the composition continues after the ocular surgery;
2.35. Method 2, or any of 2.1-2.34, wherein the method further comprises the topical ophthalmic administration of a corticosteroid;
2.36. Method 2, or any of 2.1-2.34, wherein the method does not further comprise the topical ophthalmic administration of a corticosteroid;
2.37. Method 2, or any of 2.1-2.36, wherein the method does not further comprise the topical ophthalmic administration of a drug that blocks LFA-1 binding to ICAM-1 (e.g., lifitegrast);
2.38. Method 2, or any of 2.1-2.37, wherein the composition is the only topical ophthalmic composition administered to the patient during the period when the method is carried out;
2.39. Method 2, or any of 2.1-2.38, wherein the patient has not been previously diagnosed with dry eye disease prior to evaluation for the ocular surgery;
2.40. Method 1, or any of 2.1-1.38, wherein the patient has been previously diagnosed with dry eye disease prior to evaluation for the ocular surgery;
2.41. Method 2, or any of 2.1-2.40, wherein the patient has ocular surface disease (e.g., corneal surface damage, tear film deficiency, and/or tear film disruption);
2.42. Method 2, or any of 2.1-2.41, wherein the patient is first identified as having ocular surface disease (e.g., corneal surface damage, tear film deficiency, and/or tear film disruption), in evaluation for or in preparation for the ocular surgery;
2.43. Method 2, or any of 2.1-2.41, wherein the method further comprises the step of measuring or evaluating the degree of ocular surface disease, prior to administering the composition;
2.44. Method 2, or any of 2.1-2.43, wherein the method comprises the step of determining the degree of ocular surface disease (e.g., corneal surface damage, tear film deficiency, and/or tear film disruption), after the completion of the administering of the composition (e.g., after administration of the composition for at least a period of 7 days, or for at least 14 days, or for at least 21 days, or for at least 28 days or for at least 35 days or for at least 42 days), e.g., by total corneal fluorescein staining score and/or central corneal staining score, such as for the purpose of comparison to the pre-treatment condition;
2.45. Method 2.44, wherein the method further comprises the step of comparing the degree of ocular surface disease (e.g., corneal surface damage, tear film deficiency, and/or tear film disruption) before and after the administration of the composition (e.g., before versus after administration of the composition for at least a period of 7 days, or for at least 14 days, or for at least 21 days, or for at least 28 days or for at least 35 days or for at least 42 days) to determine whether the patient can proceed with the biometry measurements and/or the ocular surgery;
2.46. Method 2, or any of 2.1-2.45, wherein the method further comprises the step of subjecting the patient to ocular surgery upon having a determination made that the ocular surface disease (e.g., corneal surface damage, tear film deficiency, and/or tear film disruption) has been reduced to a level safe and suitable for taking biometry measurements and/or performing the ocular surgery;
2.47. Method 2, or any of 2.1-2.46, wherein, before the beginning of the administration of the composition according to the method, the patient is characterized by the eye intended for ocular surgery having:
   (a) a total corneal fluorescein staining score of greater than or equal to 7 (e.g., greater than 8, 9, 10, 11, or 12), or of greater than or equal to 10 (e.g., greater than 11, 12, or 13), such as 7-15, 7-13,10-15 or 10-13; and/or
   (b) a central corneal fluorescein staining score of equal or greater than 1 (e.g., greater than 1, greater than 2), such as 1-2 or 1-3 or 2-3;
2.48. Method 2, or any of 2.1-2.47, wherein after the period of treatment with the composition (e.g., after administration of the composition for at least a period of 7 days, or for at least 14 days, or for at least 21 days, or for at least 28 days or for at least 35 days or for at least 42 days) according to the method, the patient is characterized by the eye intended for ocular surgery having:
   (a) a total corneal fluorescein staining score of less than 7 (e.g., less than or equal to 1, 2, 3, 4, 5, or 6) or less than 10 (e.g., less than or equal to 3, 4, 5, 6, 7, 8 or 9); and/or a total corneal fluorescein staining score that has decreased by at least 3 grades from the pre-treatment score; and/or
   (b) a central corneal fluorescein staining score of less than or equal to 1; and/or a central corneal fluorescein staining score that has decreased by at least 1 grade from the pre-treatment score;
2.49. Method 2, or any of 2.1-2.48, wherein the method improves the patient's total corneal fluorescein staining score in the eye intended for ocular surgery by at least 3 grades before the biometry measurements are performed and/or before the ocular surgery is performed.
2.50. Method 2, or any of 2.1-2.49, wherein the method improves the patient's central corneal fluorescein staining score in the eye intended for ocular surgery by at least 1 grade before the biometry measurements are performed and/or before the ocular surgery is performed;
2.51. Method 2, or any of 2.1-2.50, wherein the method further comprises the step of subjecting the patient to biometry measurements upon reduction of the patient's total corneal fluorescein staining score in the eye intended for ocular surgery by at least 3 grades;
2.52. Method 2, or any of 2.1-2.51, wherein the method further comprises the step of subjecting the patient to ocular surgery upon reduction of the patient's total corneal fluorescein staining score in the eye intended for ocular surgery by at least 3 grades;
2.53. Method 2, or any of 2.1-2.52, wherein the method improves the patient's tear quality in the eye intended for surgery, e.g., as shown by an improved tear film break-up time (TFBUT) and/or an improved Schirmer's Test score;
2.54. Any of Methods 2.48-2.53, wherein the patient achieves the stated level of improvement after less than 8 weeks of treatment, e.g., after 1 to 6 weeks of administration of the composition, or after 1 to 5 weeks of administration of the composition, or after 1 to 4 weeks of administration of the composition, or after 2 to 6 weeks of administration of the composition, or after 2 to 5 weeks of administration of the composition, or after 2 to 4 weeks of administration of the composition, or after 2 to 3 weeks of administration of the composition or after less than 4 weeks of administration of the composition;
2.55. Method 2, or any of 2.1-2.54, wherein the patient is subjected to biometry after the administration of the composition for sufficient time to afford a reduction at least 3 grades in the tCFS score in the eye intended for ocular surgery;
2.56. Method 2, or any of 2.1-2.55, wherein the composition is administered to the patient until the patient's total corneal fluorescein staining score in the eye intended for ocular surgery is reduced by at least 3 grades;
2.57. Method 2, or any of 2.1-2.56, wherein the patient suffers from dry eye disease at the time of evaluation or preparation for the ocular surgery, optionally, wherein the patient was not diagnosed with dry eye disease prior to the time of evaluation or preparation for the ocular surgery;
2.58. Method 2.57, wherein the patient suffers from ocular surface disease (e.g., corneal surface damage, tear film deficiency and/or tear film disruption) caused by the dry eye disease;
2.59. Method 2, or any of 2.1-2.57, wherein the patient suffers from ocular surface disease (e.g., corneal surface damage, tear film deficiency and/or tear film disruption) not caused by dry eye disease;
2.60. Method 2.59, wherein the ocular surface disease (e.g., corneal surface damage, tear film deficiency and/or tear film disruption) is caused by corneal abrasion, ocular chemical irritation, irritation due to contact lens wear (e.g., improper contact lens wear or sensitivity to contact lens care solutions), foreign body exposure, ultraviolet light injury, or ionizing radiation injury;
2.61. Method 2, or any of 2.1-2.60, wherein the patient does not suffer from Meibomian gland dysfunction;
2.62. Method 2, or any of 2.1-2.61, wherein the patient does not suffer from blepharitis or uveitis;
2.63. Method 2, or any of 2.1-2.62, wherein the composition does not comprise latanoprost, tafluprost, bimatoprost, travoprost, unoprostone, tacrolimus, sirolimus, everolimus, timolol, nebivolol, antibacterial agents, antifungal agents, antiviral agents.

In a third aspect, the present disclosure provides a method (Method 3) for performing biometry measurements for ocular surgery (e.g., cataract surgery) comprising topically administering once to four times (e.g., two times) daily to the eye of the patient a composition comprising about 0.05-0.1% (w/v) cyclosporine dissolved in a semifluorinated alkane (e.g., 1-perfluorobutylpentane), optionally with an alcoholic co-solvent (e.g., ethanol), prior to the biometry measurements.

In further embodiments of the third aspect, the present disclosure provides:
3.1. Method 3, wherein the ocular surgery is selected from cataract surgery, refractive surgery (including keratomileusis, keratoplasty, radial keratotomy, and intraocular lens implantation), glaucoma surgery (including canaloplasty, laser trabeculoplasty, iridotomy, iridectomy, sclerostomy, trabeculectomy, and viscocanalostomy), laser eye surgery (e.g., laser-assisted in situ keratomileusis or LASIK, and photorefractive keratectomy, also known as LASEK, laser-assisted epithelial keratomileusis), and vitreoretinal surgery (e.g., vitrectomy, retinal detachment repair);
3.2. Method 3.1, wherein the ocular surgery is cataract surgery or refractive surgery;
3.3. Method 3.2, wherein the ocular surgery is cataract surgery or refractive surgery via intraocular lens implantation;
3.4. Method 3, or any of 3.1-3.3, wherein the method further comprises the step of determining the degree of ocular surface disease (e.g., corneal surface damage, tear film deficiency, and/or tear film disruption) in preparation for the ocular surgery;
3.5. Method 3.4, wherein the determination of the degree of ocular surface disease comprises the step of determining the degree of corneal surface damage and/or the tear film deficiency or disruption;
3.6. Method 3.5, wherein the degree of corneal surface damage is determined by the total corneal fluorescein staining score and/or the central corneal fluorescein staining score, and/or wherein the degree of tear film deficiency or disruption is determined by the tear film breakup time (TFBUT) or Schirmer's test;
3.7. Method 3, or any of 3.1-3.6, wherein the biometry measurements comprise keratometry and/or optical biometry, such as, measurement of anterior corneal curvature, posterior corneal curvature, axial length, anterior chamber depth, lens thickness, and/or corneal power, of the eye undergoing ocular surgery;
3.8. Method 3, or any of 3.1-3.7, wherein the semifluorinated alkane is 1-perfluorobutyl-pentane;
3.9. Method 3, or any of 3.1-3.8, wherein the composition comprises 1-perfluorobutylpentane and 2-perfluorobutylpentane;
3.10. Any of Methods 3.8-3.9, wherein the composition comprises 1-perfluorobutylpentane and 2-perfluorobutylpentane, in a weight ratio of about 95:5, or about 96:4, or about 97:3, or about 98:2, or about 99:1, or about 99.5:0.55, or about 99.6:0.4, or about 99.7:0.3, or about 99.8:0.22, or about 99.9:0.1;
3.11. Any of Methods 3.8-3.10 wherein the composition comprises up to 2% (w/w) of 2-perfluorobutylpentane, or up to 1% (w/w), or up to 0.5% (w/w), or up to 0.2% (w/w), or up to 0.1% (w/w) of 2-perfluorobutylpentane, optionally wherein the composition comprises at least 0.01% (w/w) or at least 0.1% (w/w) of 2-perfluorobutylpentane;
3.12. Method 3, or any of 3.1-3.11, wherein the composition is free or substantially free of water, e.g., less than 2 wt. %, or less than 1 wt. %, or less than 0.5 wt. %, or less than 0.2 wt. %, or less than 0.1 wt. % water by weight of the composition;
3.13. Method 3, or any of 3.1-3.12, wherein the composition is free or substantially free of any preservatives, e.g., less than 3 wt. %, or less than 2 wt. %, or less than 1 wt. %, or less than 0.5 wt. %, or less than 0.1 wt. % preservatives by weight of the composition, or wherein the composition is not formulated to include any preservatives;
3.14. Method 3, or any of 3.1-3.13, wherein the composition further comprises an alcoholic co-solvent;
3.15. Method 3.14, wherein the alcoholic co-solvent is ethanol;
3.16. Any of Methods 3.14-3.15, wherein the composition comprises the alcoholic co-solvent in an amount of 0.01 to 5% (w/w), or 0.05 to 4% (w/w), or 0.1 to 3% (w/w), or 0.2 to 2% (w/w), or 0.5 to 2% (w/w), or 0.5 to 1.5% (w/w), or 0.8 to 1.2% (w/w), or about 1% (w/w), or about 1.0% (w/w);
3.17. Method 3, or any of 3.1-3.16, wherein the composition comprises about 0.05-0.1% (w/v) cyclosporine dissolved in 1-perfluorobutylpentane;
3.18. Method 3.17, wherein the composition comprises about 0.1% (w/v) cyclosporine dissolved in 1-perfluorobutylpentane;
3.19. Method 3.18, wherein the composition comprises or essentially consists of about 0.1% (w/v) cyclosporine dissolved in 1-perfluorobutylpentane with about 1% (w/w) ethanol;
3.20. Method 3.19, wherein the composition comprises or essentially consists of about 0.1% (w/v) cyclosporine dissolved in 1-perfluorobutylpentane with about 1% (w/w) ethanol and up to about 2% (w/w), (or up to about 1% (w/w) 2-perfluorobutylpentane;
3.21. Method 3.20, wherein the composition consists of about 0.1% (w/v) cyclosporine dissolved in 1-perfluorobutylpentane with about 1% (w/w) ethanol and up to about 2% (w/w), or up to about 0.5% (w/w) 2-perfluorobutylpentane;
3.22. Method 3, or any of 3.1-3.21, wherein the composition is administered four times daily;
3.23. Method 3, or any of 3.1-3.21, wherein the composition is administered three times daily;
3.24. Method 3, or any of 3.1-3.21, wherein the composition is administered two times daily;
3.25. Method 3, or any of 3.1-3.21, wherein the composition is administered once daily;
3.26. Method 3, or any of 3.1-3.25, wherein the amount of cyclosporine administered in a single dose per eye is about 8-12 μg, e.g., 9-11 μg, 9-10 μg, or about 9 μg, or about 10 μg cyclosporine;
3.27. Method 3, or any of 3.1-3.26, wherein the volume administered in a single dose per eye is about 8-12 μL, e.g., 9-11 μL, e.g., 9-10 μL, or about 9 μl, or about 10 μL;
3.28. Method 3, or any of 3.1-3.27, wherein the patient is administered the composition for at least a period of 7 days, or for at least 14 days, or for at least 21 days, or for at least 28 days or for at least 35 days; or for at least 42 days;
3.29. Method 3, or any of 3.1-1.28, wherein the step of determining the degree of ocular surface disease (e.g., corneal surface damage, tear film deficiency and/or tear film disruption) precedes the step of performing biometry measurements;
3.30. Method 3, or any of 3.1-3.29, wherein the last day of administration of the composition precedes the patient's biometry measurements, e.g., by at least 1 day, or at least 2 days, or at least 3 days, or at least 5 days, or at least 7 days, or 1-7 days;
3.31. Method 3, or any of 3.1-3.31, wherein the step of determining the degree of ocular surface disease (e.g., corneal surface damage, tear film deficiency and/or tear film disruption) precedes the administration of the composition;
3.32. Method 3, or any of 3.1-3.31, wherein the administration of the composition continues after the biometry measurement, optionally until 1-3 days before the patient's ocular surgery;
3.33. Method 3, or any of 3.1-3.32, wherein the administration of the composition continues after the ocular surgery;
3.34. Method 3, or any of 3.1-3.33, wherein the method further comprises the topical ophthalmic administration of a corticosteroid;
3.35. Method 3, or any of 3.1-3.33, wherein the method does not further comprise the topical ophthalmic administration of a corticosteroid;
3.36. Method 3, or any of 3.1-3.35, wherein the method does not further comprise the topical ophthalmic administration of a drug that blocks LFA-1 binding to ICAM-1 (e.g., lifitegrast);
3.37. Method 3, or any of 3.1-3.36, wherein the composition is the only topical ophthalmic composition administered to the patient during the period when the method is carried out;
3.38. Method 3, or any of 3.1-3.37, wherein the patient has not been previously diagnosed with dry eye disease prior to evaluation for the ocular surgery;
3.39. Method 3, or any of 3.1-3.37, wherein the patient has been previously diagnosed with dry eye disease prior to evaluation for the ocular surgery;
3.40. Method 3, or any of 3.1.-3.39, wherein the patient has ocular surface disease (e.g., corneal surface damage, tear film deficiency, and/or tear film disruption);
3.41. Method 3, or any of 3.1-3.40, wherein the patient is first identified as having ocular surface disease (e.g., corneal surface damage, tear film deficiency, and/or tear film disruption), in evaluation for or in preparation for the ocular surgery;
3.42. Method 3, or any of 3.1-3.41, wherein the method further comprises the step of measuring or evaluating the degree of ocular surface disease, prior to administering the composition;
3.43. Method 3, or any of 3.1-3.42, wherein the method comprises the step of determining the degree of ocular surface disease (e.g., corneal surface damage, tear film deficiency, and/or tear film disruption), after the completion of the administering of the composition (e.g., after administration of the composition for at least a period of 7 days, or for at least 14 days, or for at least 21 days, or for at least 28 days or for at least 35 days or for at least 42 days), e.g., by total corneal fluorescein staining score and/or central corneal fluorescein staining score, such as for the purpose of comparison to the pre-treatment condition;
3.44. Method 3.44, wherein the method further comprises the step of comparing the degree of ocular surface disease (e.g., corneal surface damage, tear film deficiency, and/or tear film disruption) before and after the administration of the composition (e.g., before versus after administration of the composition for at least a period of 7 days, or for at least 14 days, or for at least 21 days, or for at least 28 days or for at least 35 days or for at least 42 days) to determine whether the patient can proceed with the biometry measurements and/or the ocular surgery;
3.45. Method 3, or any of 3.1-3.44, wherein, before the beginning of the administration of the composition according to the method, the patient is characterized by the eye intended for ocular surgery having:
   (a) a total corneal fluorescein staining score of greater than or equal to 7 (e.g., greater than 8, 9, 10, 11, or 12), or greater than or equal to 10 (e.g., greater than 11, 12, or 13), such as 7-15, 7-13,10-15 or 10-13;
   (b) a central corneal fluorescein staining score of equal or greater than 1 (e.g., greater than 1 or greater than 2), such as 1-2 or 1-3 or 2-3; and/or
3.46. Method 3, or any of 3.1-3.45, wherein after the period of treatment with the composition (e.g., after administration of the composition for at least a period of 7 days, or for at least 14 days, or for at least 21 days, or for at least 28 days or for at least 35 days or for at least 42 days) according to the method, the patient is characterized by the eye intended for ocular surgery having:
   (a) a total corneal fluorescein staining score of less than 7 (e.g., less than or equal to 1, 2, 3, 4, 5, or 6), or of less than 10 (e.g., less than or equal to 3, 4, 5, 6, 7, 8 or 9); and/or a total corneal fluorescein staining score that that has decreased by at least 3 grades from the pre-treatment score; and/or
   (b) a central corneal fluorescein staining score of less than or equal to 1; and/or a central corneal fluorescein staining score that has decreased by at least 1 grade from the pre-treatment score;
3.47. Method 3, or any of 3.1-3.45, wherein the method improves the patient's total corneal fluorescein staining score in the eye intended for ocular surgery by at least 3 grades before the biometry measurements are performed and/or before the ocular surgery is performed.
3.48. Method 3, or any of 3.1-3.47, wherein the method improves the patient's central corneal fluorescein staining score in the eye intended for ocular surgery by at least 1 grade before the biometry measurements are performed and/or before the ocular surgery is performed;
3.49. Method 3, or any of 3.1-3.48, wherein the biometry measurements are performed upon reduction of the patient's total corneal fluorescein staining score in the eye intended for ocular surgery by at least 3 grades;
3.50. Method 3, or any of 3.1-3.49, wherein the method further comprises the step of subjecting the patient to ocular surgery upon reduction of the patient's total corneal fluorescein staining score in the eye intended for ocular surgery by at least 3 grades;
3.51. Method 3, or any of 3.1-3.50, wherein the method improves the patient's tear quality in the eye intended for surgery, e.g., as shown by an improved tear film break-up time (TFBUT) and/or an improved Schirmer's Test score;
3.52. Any of Methods 3.46-3.51, wherein the patient achieves the stated level of improvement after less than 8 weeks of treatment, e.g., after 1 to 6 weeks of administration of the composition, or after 1 to 5 weeks of administration of the composition, or after 1 to 4 weeks of administration of the composition, or after 2 to 6 weeks of administration of the composition, or after 2 to 5 weeks of administration of the composition, or after 2 to 4 weeks of administration of the composition, or after 2 to 3 weeks of administration of the composition or after less than 4 weeks of administration of the composition;
3.53. Method 3, or any of 3.1-3.52, wherein the patient is subjected to biometry after the administration of the composition for sufficient time to afford a reduction at least 3 grades in the tCFS score in the eye intended for ocular surgery;
3.54. Method 3, or any of 3.1-3.53, wherein the composition is administered to the patient until the patient's total corneal fluorescein staining score in the eye intended for ocular surgery is reduced by at least 3 grades;
3.55. Method 3, or any of 3.1-3.54, wherein the patient suffers from dry eye disease at the time of evaluation or preparation for the ocular surgery, optionally, wherein the patient was not diagnosed with dry eye disease prior to the time of evaluation or preparation for the ocular surgery;
3.56. Method 3.55, wherein the patient suffers from ocular surface disease (e.g., corneal surface damage, tear film deficiency and/or tear film disruption) caused by the dry eye disease;
3.57. Method 3, or any of 3.1-3.55, wherein the patient suffers from ocular surface disease (e.g., corneal surface damage, tear film deficiency and/or tear film disruption) not caused by dry eye disease;
3.58. Method 3.57, wherein the ocular surface disease (e.g., corneal surface damage, tear film deficiency and/or tear film disruption) is caused by corneal abrasion, ocular chemical irritation, irritation due to contact lens wear (e.g., improper contact lens wear or sensitivity to contact lens care solutions), foreign body exposure, ultraviolet light injury, or ionizing radiation injury;
3.59. Method 3, or any of 3.1-3.58, wherein the patient does not suffer from Meibomian gland dysfunction;
3.60. Method 3, or any of 3.1-3.59, wherein the patient does not suffer from blepharitis or uveitis;
3.61. Method 3, or any of 3.1-3.60, wherein the composition does not comprise latanoprost, tafluprost, bimatoprost, travoprost, unoprostone, tacrolimus, sirolimus, everolimus, timolol, nebivolol, antibacterial agents, antifungal agents, antiviral agents.
3.62. Method 3, or any of 3.1-3.61, wherein the administration of the composition improves the ocular surface for more accurate biometry measurements.

The present disclosure also relates to embodiments described according to the following items:
1. A method for preparing a patient for ocular surgery (e.g., cataract surgery), the method comprising topically administering two times daily to the eye of the patient (e.g., the eye undergoing ocular surgery) a composition comprising about 0.1% (w/v) cyclosporine dissolved in 1-perfluorobutylpentane, optionally with about 1% (w/w) ethanol.
2. A method for treating corneal surface damage in a patient preparing for ocular surgery (e.g., cataract surgery), the method comprising topically administering two times daily to the eye of the patient a composition comprising about 0.1% (w/v) cyclosporine dissolved in 1-perfluorobutylpentane, optionally with about 1% (w/w) ethanol.
3. The method according to any one of items 1-2, wherein the method further comprises the step of performing biometry measurements in preparation for the ocular surgery.
4. A method for performing biometry measurements for ocular surgery (e.g., cataract surgery) comprising topically administering two times daily to the eye of the patient a composition comprising about 0.1% (w/v) cyclosporine dissolved in 1-perfluorobutylpentane, optionally with about 1% (w/w) ethanol, prior to the biometry measurements.
5. The method according to any one of items 1-4, wherein the composition comprises about 0.1% (w/v) cyclosporine dissolved in 1-perfluorobutylpentane with about 1% (w/w) ethanol, and optionally up to about 1% (w/w) 2-perfluorobutylpentane.
6. The method according to any one of items 1-5, wherein the composition consists of about 0.1% (w/v) cyclosporine dissolved in 1-perfluorobutylpentane with about 1% (w/w) ethanol, and optionally up to about 0.5% (w/w) 2-perfluorobutylpentane.
7. The method according to any one of items 1-6, wherein the amount of cyclosporine administered in a single dose per eye is about 8-12 μg, e.g., 9-11 μg, or about 10 μg cyclosporine.
8. The method according to any one of items 1-7, wherein the patient is administered the composition for at least a period of 7 days, or for at least 14 days, or for at least 21 days, or for at least 28 days or for at least 35 days or for at least 42 days.
9. The method according to any of the items 1-8, wherein the method further comprises the step of determining the degree of corneal surface damage in preparation for the ocular surgery.
10. The method according to item 9, wherein the degree of corneal surface damage is determined by the total corneal fluorescein staining score and/or the central corneal fluorescein staining score.
11. The method according to any one of items 1-10, wherein, before the beginning of the administration of the composition according to the method, the patient is characterized by the eye intended for ocular surgery having:
   (a) a total corneal fluorescein staining score of greater than or equal to 7 (e.g., greater than 8, 9, 10, 11, or 12), such as 7-15, 7-13, 10-15 or 10-13;
   (b) a central corneal fluorescein staining score of equal or greater than 1 (e.g., greater than 1, greater than 2), such as 1-2 or 1-3 or 2-3.
12. The method according to any one of items 1-11, wherein after the period of treatment with the composition (e.g., after administration of the composition for at least a period of 7 days, or for at least 14 days, or for at least 21 days, or for at least 28 days or for at least 35 days or for at least 42 days) according to the method, the patient is characterized by the eye intended for ocular surgery having:
   (a) a total corneal fluorescein staining score that has decreased by at least 3 grades; and/or
   (b) a central corneal fluorescein staining score that has decreased by at least 1 grade.
13. The method according to any one of items 1-12, wherein the method improves the patient's total corneal fluorescein staining score in the eye intended for ocular surgery by at least 3 grades before the biometry measurements are performed and/or before the ocular surgery is performed.
14. The method according to any one of items 1-13, wherein the method improves the patient's central corneal fluorescein staining score in the eye intended for ocular surgery by at least 1 grade before the biometry measurements are performed and/or before the ocular surgery is performed.
15. The method according to items 1 to 14, wherein the step of determining the degree of corneal surface damage precedes the administration of the composition.
16. The method according to items 1 to 15, wherein the patient is subjected to biometry not before a reduction of the patient's total corneal fluorescein staining score in the eye intended for ocular surgery by at least 3 grades is achieved.
17. The method according to items 1 to 16, wherein the composition is administered to the patient until a reduction of the patient's total corneal fluorescein staining score in the eye intended for ocular surgery by at least 3 grades is achieved.
18. The method according to items 1 to 17, wherein the patient suffers from corneal surface damage not caused by dry eye disease.
19. The method according to items 1 to 18, wherein the administration of the composition continues after the ocular surgery.

In a fourth aspect, the present disclosure provides for the use of a composition comprising about 0.05-0.1% (w/v) cyclosporine dissolved in a semifluorinated alkane, optionally with an alcoholic co-solvent, for carrying out the method according to any of Method 1 et seq., Method 2 et seq., or Method 3 et seq.

In a fifth aspect, the present disclosure provides a pharmaceutical composition comprising about 0.05-0.1% (w/v) cyclosporine dissolved in a semifluorinated alkane, optionally with an alcoholic co-solvent, or a pharmaceutical composition as defined in any one, or combination of the embodiments described in the present disclosure, for use in a method according to any of Method 1 et seq., Method 2 et seq., or Method 3 et seq. For example, the present disclosure provides a pharmaceutical composition for use in: (a) preparing a patient for ocular surgery (e.g., cataract surgery), or (b) treating ocular surface disease (e.g. corneal surface damage, tear film deficiency and/or tear film disruption) in a patient preparing for ocular surgery (e.g., cataract surgery), or (c) performing biometry measurements in preparation for ocular surgery (e.g., cataract surgery), wherein the composition comprises about 0.05-0.1% (w/v) cyclosporine dissolved in a semifluorinated alkane, optionally with an alcoholic co-solvent, and is formulated for topical administration to the eye of the patient.

Another embodiment according to this aspect of the present disclosure relates to a composition comprising about 0.05-0.1% (w/v) cyclosporine dissolved in a semifluorinated alkane, optionally with an alcoholic co-solvent (e.g. ethanol), for use in:
a) a method of preparing a patient for ocular surgery (e.g., cataract surgery); and/or
b) a method in treating ocular surface disease (e.g., corneal surface damage, tear film deficiency or tear film disruption), in a patient preparing for ocular surgery (e.g., cataract surgery); and/or
c) a method for performing biometry measurements for ocular surgery (e.g., cataract surgery) wherein the composition is topically administered to the eye of a patient prior to the biometry measurements;
wherein the composition is topically administered once to four times daily (e.g. two times daily) to the eye of the patient (e.g., the eye of the patient undergoing ocular surgery, e.g. cataract surgery).

Further embodiments relating to said composition for use in the methods and treatments of a), b) or c) provides:
5.1. The composition for use, wherein the ocular surgery is selected from cataract surgery, refractive surgery (including keratomileusis, keratoplasty, radial keratotomy, and intraocular lens implantation), glaucoma surgery (including canaloplasty, laser trabeculoplasty, iridotomy, iridectomy, sclerostomy, trabeculectomy, and viscocanalostomy), laser eye surgery (e.g., laser-assisted in situ keratomileusis or LASIK, and photorefractive keratectomy, also known as LASEK, laser-assisted epithelial keratomileusis), and vitreoretinal surgery (e.g., vitrectomy, retinal detachment repair);
5.2. The composition for use according to item 5.1, wherein the ocular surgery is cataract surgery or refractive surgery;
5.3. The composition for use, or according to item 5.2, wherein the ocular surgery is cataract surgery or refractive surgery via intraocular lens implantation;
5.4. The composition for use, or any of items 5.1-5.3, wherein the method further comprises the step of determining the degree of ocular surface disease (e.g., corneal surface damage, tear film deficiency, and/or tear film disruption) in preparation for the ocular surgery;
5.5. The composition for use, or according to item 5.4, wherein the determination of the degree of ocular surface disease comprises the step of determining the degree of corneal surface damage and/or tear film deficiency or disruption;
5.6. The composition for use, or according to item 5.5, wherein the degree of corneal surface damage is determined by the total corneal fluorescein staining score and/or the central corneal fluorescein staining score, and/or wherein the degree of tear film deficiency or disruption is determined by the tear film breakup time (TFBUT) or Schirmer's test;
5.7. The composition for use, or according to any of items 5.1-5.6, wherein the method further comprises the step of performing biometry measurements in preparation for the ocular surgery;
5.8. The composition for use, or according to item 5.7, wherein the biometry measurements comprise keratometry and/or optical biometry, such as, measurement of corneal curvature (e.g., anterior corneal curvature, posterior corneal curvature), axial length, anterior chamber depth, lens thickness, and/or corneal power, of the eye undergoing ocular surgery;
5.9. The composition for use, or according to any of items 5.1-5.8, wherein the semifluorinated alkane is 1-perfluorobutylpentane;
5.10. The composition for use, or according to any of items 5.1-5.9, wherein the composition comprises 1-perfluorobutylpentane and 2-perfluorobutylpentane;
5.11. The composition for use, or according to any of items 5.9-5.10, wherein the composition comprises 1-perfluorobutylpentane and 2-perfluorobutylpentane, in a weight ratio of about 95:5, or about 96:4, or about 97:3, or about 98:2, or about 99:1, or about 99.5:0.5, or about 99.6:0.4, or about 99.7:0.3, or about 99.8:0.2, or about 99.9:0.1;
5.12. The composition for use, or according to any of items 5.9-5.11, wherein the composition comprises up to 5% (w/w) of 2-perfluorobutylpentane, or up to 3% (w/w), or up to 2% (w/w), or up to 1% (w/w), or up to 0.5% (w/w), or up to 0.4% (w/w), or up to 0.3% (w/w), or up to 0.2% (w/w), or up to 0.1% (w/w) of 2-perfluorobutylpentane, optionally wherein the composition comprises at least 0.01% (w/w) or at least 0.1% (w/w) or at least 0.3% (w/w) of 2-perfluorobutylpentane;
5.13. The composition for use, or according to any of items 5.1-5.12, wherein the composition is free or substantially free of water, e.g., less than 2 wt. %, or less than 1 wt. %, or less than 0.5 wt. %, or less than 0.2 wt. %, or less than 0.1 wt. % water by weight of the composition;
5.14. The composition for use, or according to any of items 5.1-5.13, wherein the composition is free or substantially free of any preservatives, e.g., less than 3 wt. %, or less than 2 wt. %, or less than 1 wt. %, or less than 0.5 wt. %, or less than 0.1 wt. % preservatives by weight of the composition, or wherein the composition is not formulated to include any preservatives;
5.15. The composition for use, or according to any of items 5.1-5.14, wherein the composition further comprises an alcoholic co-solvent;
5.16. The composition for use, or according to item 5.15, wherein the alcoholic co-solvent is ethanol;
5.17. The composition for use, or according to any of items 5.15-5.16, wherein the composition comprises the alcoholic co-solvent in an amount of 0.01 to 5% (w/w), or 0.05 to 4% (w/w), or 0.1 to 3% (w/w), or 0.2 to 2% (w/w), or 0.2 to 1.5% (w/w), or 0.2 to 1.0% (w/w), or 0.5 to 1.2% (w/w), or about 0.5% (w/w), or about 1.0% (w/w);
5.18. The composition for use, or according to any of items 5.1-5.17, wherein the composition comprises about 0.05-0.1% (w/v) cyclosporine dissolved in 1-perfluorobutylpentane;
5.19. The composition for use, or according to item 5.18, wherein the composition comprises about 0.1% (w/v) cyclosporine dissolved in 1-perfluorobutylpentane;
5.20. The composition for use, or according to item 5.19, wherein the composition comprises or essentially consists of about 0.1% (w/v) cyclosporine dissolved in 1-perfluorobutylpentane with about 1% (w/w) ethanol;
5.21. The composition for use, or according to item 5.20, wherein the composition comprises or essentially consists of about 0.1% (w/v) cyclosporine dissolved in 1-perfluorobutylpentane with about 1% (w/w) ethanol and up to about 1% (w/w) 2-perfluorobutylpentane;
5.22. The composition for use, or according to item 5.21, wherein the composition consists of about 0.1% (w/v) cyclosporine dissolved in 1-perfluorobutylpentane with about 1% (w/w) ethanol and up to about 0.5% (w/w) 2-perfluorobutylpentane;
5.23. The composition for use, or according to any of items 5.1-5.22, wherein the composition is administered four times daily;
5.24. The composition for use, or according to any of items 5.1-5.22, wherein the composition is administered three times daily;
5.25. The composition for use, or according to any of items 5.1-5.22, wherein the composition is administered two times daily;
5.26. The composition for use, or according to any of items 5.1-5.22, wherein the composition is administered once daily;
5.27. The composition for use, or according to any of items 5.1-5.26, wherein the amount of cyclosporine administered in a single dose per eye is about 8-12 μg, e.g., 9-11 μg, 9-10 μg, or about 9 μg, or about 10 μg cyclosporine;
5.28. The composition for use, or according to any of items 5.1-5.27, wherein the volume administered in a single dose per eye is about 8-12 μL, e.g., 9-11 μL, e.g., 9-10 μL, or about 9 μl or about 10 μL;
5.29. The composition for use, or according to any of items 5.1-5.28, wherein the patient is administered the composition for at least a period of 7 days, or for at least 14 days, or for at least 21 days, or for at least 28 days, or for at least 35 days; or for at least 42 days;
5.30. The composition for use, or according to any of items 5.1-5.29, wherein the step of determining the degree of ocular surface disease (e.g., corneal surface damage, tear film efficiency and/or tear film disruption) precedes the step of performing biometry measurements;
5.31. The composition for use, or according to any of items 5.1-5.30, wherein the last day of administration of the composition precedes the patient's biometry measurements, e.g., by at least 1 day, or at least 2 days, or at least 3 days, or at least 5 days, or at least 7 days, or 1-7 days;
5.32. The composition for use, or according to any of items 5.1-5.31, wherein the step of determining the degree of ocular surface disease (e.g., corneal surface damage, tear film deficiency and/or tear film disruption) precedes the administration of the composition;
5.33. The composition for use, or according to any of items 5.1-5.32, wherein the administration of the composition continues after the biometry measurement, optionally until 1-3 days before the patient's ocular surgery;
5.34. The composition for use, or according to any of items 5.1-5.33, wherein the administration of the composition continues after the ocular surgery;
5.35. The composition for use, or according to any of items 5.1-5.34, wherein the method further comprises the topical ophthalmic administration of a corticosteroid;
5.36. The composition for use, or according to any of items 5.1-5.34, wherein the method does not further comprise the topical ophthalmic administration of a corticosteroid;
5.37. The composition for use, or according to any of items 5.1-5.36, wherein the method does not further comprise the topical ophthalmic administration of a drug that blocks LFA-1 binding to ICAM-1 (e.g., lifitegrast);
5.38. The composition for use, or according to any of items 5.1-5.37, wherein the composition is the only topical ophthalmic composition administered to the patient during the period when the method or treatment is carried out;
5.39. The composition for use, or according to any of items 5.1-5.38, wherein the patient has not been previously diagnosed with dry eye disease prior to evaluation for the ocular surgery;
5.40. The composition for use, or according to any of items 5.1-5.38, wherein the patient has been previously diagnosed with dry eye disease prior to evaluation for the ocular surgery;
5.41. The composition for use, or according to any of items 5.1-5.40, wherein the patient has ocular surface disease (e.g., corneal surface damage, tear film deficiency, and/or tear film disruption);
5.42. The composition for use, or according to any of items 5.1-5.41, wherein the patient is first identified as having ocular surface disease (e.g., corneal surface damage, tear film deficiency, and/or tear film disruption), in evaluation for or in preparation for the ocular surgery;
5.43. The composition for use, or according to any of items 5.1-5.41, wherein the method further comprises the step of measuring or evaluating the degree of ocular surface disease, prior to administering the composition;
5.44. The composition for use, or according to any of items 5.1-5.43, wherein the method further comprises the step of determining the degree of ocular surface disease (e.g., corneal surface damage, tear film deficiency, and/or tear film disruption), after the completion of the administering of the composition (e.g., after administration of the composition for at least a period of 7 days, or for at least 14 days, or for at least 21 days, or for at least 28 days or for at least 35 days or for at least 42 days), e.g., by total corneal fluorescein staining score and/or central corneal fluorescein staining score, such as for the purpose of comparison to the pre-treatment condition;
5.45. The composition for use, or according to item 5.44, wherein the method further comprises the step of comparing the degree of ocular surface disease (e.g., corneal surface damage, tear film deficiency, and/or tear film disruption) before and after the administration of the composition (e.g., before versus after administration of the composition for at least a period of 7 days, or for at least 14 days, or for at least 21 days, or for at least 28 days or for at least 35 days or for at least 42 days) to determine whether the patient can proceed with the biometry measurements and/or the ocular surgery;
5.46. The composition for use, or according any of items 5.1-5.45, wherein the method further comprises the step of subjecting the patient to ocular surgery upon having a determination made that the ocular surface disease (e.g., corneal surface damage, tear film deficiency, and/or tear film disruption) has been reduced to a level safe and suitable for taking biometry measurements and/or performing the ocular surgery;
5.47. The composition for use, or according to any of items 5.1-5.46, wherein, before the beginning of the administration of the composition according to the method, the patient is characterized by the eye intended for ocular surgery having:
   (a) a total corneal fluorescein staining score of greater than or equal to 7 (e.g., greater than 8, 9, 10, 11, or 12), or greater than or equal to 10 (e.g., greater than 10, 11, 12 or 13), such as 7-15, 7-13, 10-15 or 10-13; and/or
   (b) a central corneal fluorescein staining score of equal or greater than 1 (e.g., greater than 1, greater than 2), such as 1-2 or 1-3 or 2-3;
5.48. The composition for use, or according to any one of items 5.1-5.47, wherein after the period of treatment with the composition (e.g., after administration of the composition for at least a period of 7 days, or for at least 14 days, or for at least 21 days, or for at least 28 days or for at least 35 days or for at least 42 days) according to the method, the patient is characterized by the eye intended for ocular surgery having:
   (a) a total corneal fluorescein staining score of less than 7 (e.g., less than or equal to 1, 2, 3, 4, 5, or 6) or less than 10 (e.g., less than or equal to 3, 4, 5, 6, 7, 8 or 9); and/or a total corneal fluorescein staining score that has decreased by at least 3 grades from the pre-treatment score; and/or
   (b) a central corneal fluorescein staining score of less than or equal to 1; and/or a central corneal fluorescein staining score that has decreased by at least 1 grade from the pre-treatment score;
5.49. The composition for use, or according to any of items 5.1-5.48, wherein the method improves the patient's total corneal fluorescein staining score in the eye intended for ocular surgery by at least 3 grades before the biometry measurements are performed and/or before the ocular surgery is performed;
5.50. The composition for use, or according to any of items 5.1-5.49, wherein the method improves the patient's central corneal fluorescein staining score in the eye intended for ocular surgery by at least 1 grade before the biometry measurements are performed and/or before the ocular surgery is performed;
5.51. The composition for use, or according to any of items 5.1-5.50, wherein the method further comprises the step of subjecting the patient to biometry measurements upon reduction of the patient's total corneal fluorescein staining score in the eye intended for ocular surgery by at least 3 grades;
5.52. The composition for use, or according to any of items 5.1-5.51, wherein the method further comprises the step of subjecting the patient to ocular surgery upon reduction of the patient's total corneal fluorescein staining score in the eye intended for ocular surgery by at least 3 grades;
5.53. The composition for use, or according to any of items 5.1-5.52, wherein the method improves the patient's tear quality in the eye intended for surgery, e.g., as shown by an improved tear film break-up time (TFBUT) and/or an improved Schirmer's Test score;
5.54. The composition for use, or according to any of items 5.48-5.53, wherein the patient achieves the stated level of improvement after less than 8 weeks of treatment, e.g., after 1 to 6 weeks of administration of the composition, or after 1 to 5 weeks of administration of the composition, or after 1 to 4 weeks of administration of the composition, or after 2 to 6 weeks of administration of the composition, or after 2 to 5 weeks of administration of the composition, or after 2 to 4 weeks of administration of the composition, or after 2 to 3 weeks of administration of the composition or after less than 4 weeks of administration of the composition;
5.55. The composition for use, or according to any of items 5.1-5.54, wherein the patient is subjected to biometry after the administration of the composition for sufficient time to afford a reduction at least 3 grades in the tCFS score in the eye intended for ocular surgery;
5.56. The composition for use, or according to any of items 5.1-5.55, wherein the composition is administered to the patient until the patient's total corneal fluorescein staining score in the eye intended for ocular surgery is reduced by at least 3 grades;
5.57. The composition for use, or according to any of items 5.1-5.56, wherein the patient suffers from dry eye disease at the time of evaluation or preparation for the ocular surgery (e.g., cataract surgery), optionally, wherein the patient was not diagnosed with dry eye disease prior to the time of evaluation or preparation for the ocular surgery (e.g., cataract surgery);
5.58. The composition for use, or according to item 5.57 wherein the patient suffers from ocular surface disease (e.g., corneal surface damage, tear film deficiency and/or tear film disruption) caused by the dry eye disease;
5.59. The composition for use, or according to any of items 5.1-5.57, wherein the patient suffers from ocular surface disease (e.g., corneal surface damage, tear film deficiency and/or tear film disruption) not caused by dry eye disease;
5.60. The composition for use, or according to item 5.59, wherein the ocular surface disease (e.g., corneal surface damage, tear film deficiency and/or tear film disruption) is caused by corneal abrasion, ocular chemical irritation, irritation due to contact lens wear (e.g., improper contact lens wear or sensitivity to contact lens care solutions), foreign body exposure, ultraviolet light injury, or ionizing radiation injury;
5.61. The composition for use, or according to any of items 5.1-5.60, wherein the patient does not suffer from Meibomian gland dysfunction;
5.62. The composition for use, or according to any of items 5.1-5.61, wherein the patient does not suffer from blepharitis or uveitis;
5.63. The composition for use, or according to any of items 5.1-5.62, wherein the composition does not comprise latanoprost, tafluprost, bimatoprost, travoprost, unoprostone, tacrolimus, sirolimus, everolimus, timolol, nebivolol, antibacterial agents, antifungal agents, antiviral agents.
5.64. The composition for use, or according to any of items 5.1-5.63, wherein the administration of the composition improves the ocular surface for more accurate biometry measurements.

Further embodiments of this aspect according to the present disclosure provides for:
1.1. A composition comprising about 0.1% (w/v) cyclosporine dissolved in 1-perfluorobutylpentane, optionally with about 1% (w/w) ethanol for use in a method of preparing a patient for ocular surgery (e.g., cataract surgery), the method comprising topically administering the composition two times daily to the eye of the patient (e.g., the eye undergoing ocular surgery.
1.2. A composition comprising about 0.1% (w/v) cyclosporine dissolved in 1-perfluorobutylpentane, optionally with about 1% (w/w) ethanol for use in a method of treating corneal surface damage in a patient preparing for ocular surgery (e.g., cataract surgery), the method comprising topically administering the composition two times daily to the eye of the patient.
1.3. The composition for use according to any one of items 1.1-1.2, wherein the method further comprises the step of performing biometry measurements in preparation for the ocular surgery.
1.4. A composition comprising about 0.1% (w/v) cyclosporine dissolved in 1-perfluorobutylpentane, optionally with about 1% (w/w) ethanol for use in a method of performing biometry measurements for ocular surgery (e.g., cataract surgery), the method comprising topically administering the composition two times daily to the eye of the patient, prior to the biometry measurements.
1.5. The composition for use according to any one of items 1.1-1.4, wherein the composition comprises about 0.1% (w/v) cyclosporine dissolved in 1-perfluorobutylpentane with about 1% (w/w) ethanol, and optionally up to about 1% (w/w) 2-perfluorobutylpentane.
1.6. The composition for use according to any one of items 1.1-1.5, wherein the composition consists of about 0.1% (w/v) cyclosporine dissolved in 1-perfluorobutylpentane with about 1% (w/w) ethanol, and optionally up to about 0.5% (w/w) 2-perfluorobutylpentane.
1.7. The composition for use according to any one of items 1.1-1.6, wherein the amount of cyclosporine administered in a single dose per eye is about 8-12 μg, e.g., 9-11 μg, or about 10 μg cyclosporine.
1.8. The composition for use according to any one of items 1.1-1.7, wherein the patient is administered the composition for at least a period of 7 days, or for at least 14 days, or for at least 21 days, or for at least 28 days or for at least 35 days or for at least 42 days.
1.9. The composition for use according to any of the items 1.1-1.8, wherein the method further comprises the step of determining the degree of corneal surface damage in preparation for the ocular surgery.
1.10. The composition for use according to item 1.9, wherein the degree of corneal surface damage is determined by the total corneal fluorescein staining score and/or the central corneal fluorescein staining score.
1.11. The composition for use according to any one of items 1.1-1.10, wherein, before the beginning of the administration of the composition according to the method, the patient is characterized by the eye intended for ocular surgery having:
   (a) a total corneal fluorescein staining score of greater than or equal to 7 (e.g., greater than 8, 9, 10, 11, or 12), such as 7-15, 7-13, 10-15 or 10-13;
   (b) a central corneal fluorescein staining score of equal or greater than 1 (e.g., greater than 1, greater than 2), such as 1-2 or 1-3 or 2-3.
1.12. The composition for use according to any one of items 1.1-1.11, wherein after the period of treatment with the composition (e.g., after administration of the composition for at least a period of 7 days, or for at least 14 days, or for at least 21 days, or for at least 28 days or for at least 35 days or for at least 42 days) according to the method, the patient is characterized by the eye intended for ocular surgery having:
   (a) a total corneal fluorescein staining score that has decreased by at least 3 grades; and/or
   (b) a central corneal fluorescein staining score that has decreased by at least 1 grade.
1.13. The composition for use according to any one of items 1.1-1.12, wherein the method improves the patient's total corneal fluorescein staining score in the eye intended for ocular surgery by at least 3 grades before the biometry measurements are performed and/or before the ocular surgery is performed.
1.14. The composition for use according to any one of items 1.1-1.13, wherein the method improves the patient's central corneal fluorescein staining score in the eye intended for ocular surgery by at least 1 grade before the biometry measurements are performed and/or before the ocular surgery is performed.
1.15. The composition for use according to any one of items 1.1 to 1.14, wherein the step of determining the degree of corneal surface damage precedes the administration of the composition.
1.16. The composition for use according to any one of items 1.1 to 1.15, wherein the patient is subjected to biometry not before a reduction of the patient's total corneal fluorescein staining score in the eye intended for ocular surgery by at least 3 grades is achieved.
1.17. The composition for use according to any one of items 1.1 to 1.16, wherein the composition is administered to the patient until a reduction of the patient's total corneal fluorescein staining score in the eye intended for ocular surgery by at least 3 grades is achieved.
1.18. The composition for use according to any one of items 1.1 to 1.17, wherein the patient suffers from corneal surface damage not caused by dry eye disease.
1.19. The composition for use according to any one of items 1.1 to 1.18, wherein the administration of the composition continues after the ocular surgery.

In a further embodiment, the present disclosure provides a pharmaceutical composition comprising about 0.1% (w/v) cyclosporine dissolved in 1-perfluorobutylpentane, optionally with about 1% (w/w) ethanol and further optionally up to about 1% (w/w) 2-perfluorobutylpentane, for use in (a) preparing a patient for cataract surgery (or a surgery involving artificial lens implantation), or (b) for treating ocular surface disease, such as corneal surface damage, in a patient preparing for cataract surgery (or a surgery involving artificial lens implantation), wherein the method comprises topical administration of the composition to the eye of the patient, at least two times daily, preferably in an amount of between 8 to 12 μL (or 9 to 11 μL, or 10 μL) volume of the composition. In some embodiments, the composition is used for the preparation of a patient for cataract surgery, e.g. preparing an eye of the patient for biometry measurements prior to the surgery, wherein the use comprises first an evaluation for corneal surface damage of the eye, for example by corneal fluorescein staining as described herein, followed by administration of the composition as described to the eye, prior to the taking of any biometry measurements and/or the surgery. The eye (which may be one or both eyes) of the patient for which cataract or lens implantation surgery is intended and for which said composition may be used may be assessed to have corneal surface damage, e.g. having corneal fluorescein staining scores such as described in Methods 1.47 or Methods 2.47. The use of said composition comprises administering the composition to the eye intended for surgery for a treatment period which precedes the determination of biometry measurements of the eye and/or the cataract surgical procedure, optionally wherein the step of evaluating corneal surface damage may be repeated, e.g. at least once during the treatment period, and the treatment also may be continued until an improvement of total corneal fluorescein staining is observed; for example, a decrease in scoring by between 1 to 3 grades, before the next steps of biometry measurements and/or the surgery on the eye. Optionally, the use of the composition may continue after the biometry measurement and up until surgery, for example up until 1-3 days before the patient's ocular surgery.

In a further aspect, the present disclosure provides a kit comprising a pharmaceutical composition comprising about 0.05-0.1% (w/v) cyclosporine dissolved in a semifluorinated alkane, optionally with an alcoholic co-solvent, formulated for topical administration to the eye of the patient, or a pharmaceutical composition as defined in any one, or combination of the embodiments described in the present disclosure, wherein the kit comprises a container for holding the composition and a drop dispenser adapted for administering about 8-12 μL volume (or 9-11 μl, or 9-10 μl, or about 9 μl or about 10 μl) of the composition per drop, and instructions for administering the composition according to any of Method 1 et seq., Method 2 et seq., or Method 3 et seq.

As used herein, “in preparation for ocular surgery” means any pre-operative preparation for ocular surgery (such as cataract surgery), including any diagnostic testing performed in order to confirm the patient's suitability for ocular surgery or for the preparation of an artificial intraocular lens. Preparation is typically performed by, or at the direction of, an eye care provider, generally after the decision is made that ocular surgery is necessary or desirable.

As used herein, “evaluation for ocular surgery” means any diagnostic or evaluative steps taken before preparation for ocular surgery (such as cataract surgery), including diagnostic tests to determine the presence of a surgically repairable defect or the potential need and suitability of the patient for ocular surgery. Evaluation is typically performed by, or at the direction of, an ophthalmologist, and precedes the determination that cataract surgery is necessary or appropriate.

In preparation for ocular surgery, especially surgery involving artificial lens implantation, the length of the eye must be measured in what is called an A-scan, and the curvature of the cornea must be measured using a technique called keratometry. These measurements are critical in enabling a surgeon to define the proper parameters for the ocular surgery, such as selection of the proper lens implant for the eye.

Keratometry (K) is the measurement of the corneal curvature; corneal curvature determines the power of the cornea. Differences in power across the cornea (opposite meridians) results in astigmatism; therefore, keratometry measures astigmatism. It can be acquired with a variety of instruments either manually or via automated methods.

Measurements can be very sophisticated, such as with topographers, that measure a cornea across a broad number of points, or it can be measured in a more finite area of the cornea, for example with a manual keratometer or with the IOL Master (Carl Zeiss Meditec). The IOL Master also measures the axial length and other ocular parameters (such as anterior chamber depth and white-to-white measurements) and includes K readings.

Keratometry is a particularly critical measurement because any errors in measurements directly result in reduced refractive outcomes. Thus, if K is inaccurate, the patient will have an unexpected refractive surprise post-operatively, and such a refractive mis-outcome is very disappointing to the doctor and the patient. Correcting such errors often requires a second surgical procedure, such as exchanging out a new intraocular lens, piggy-backing of an additional intraocular lens, or a surface adjustment procedure.

Optical biometry is the process of measuring the corneal power and length of the eye. Inaccuracy in either of these measurements will also lead to an unpredicted postoperative refractive error. Optical biometry is the current standard for intraocular lens (IOL) power calculations in clinical practice. Optical biometry is a highly accurate non-invasive automated method for measuring the anatomical characteristics of the eye. Accurate measurements are critical for determining the correct power of an intraocular lens before it is implanted.

Optical biometry, also known as ophthalmic biometry, using partial coherence interferometry has become the gold standard in ocular biometry as it is highly accurate, easy to perform, non-invasive and comfortable for the patient.

Four major components are determined in optical biometry measurements:
(1) corneal curvature (keratometry),
(2) axial length (AL),
(3) anterior chamber depth (ACD) and
(4) optionally, lens thickness (LT).

These are the four key variables that are utilized to calculate the power for an artificial intraocular lens.

The anterior surface of the cornea can be measured using a variety of technologies, including reflection-based approaches such as keratometry and corneal topography, Scheimpflug imaging and anterior segment OCT. Each technology has its own set of advantages and disadvantages. Reflection-based methods can measure with great precision, but they have limits when it comes to obtaining information on the posterior corneal surface and even ultrastructural information from the cornea. Scheimpflug measures and anterior segment OCT, on the other hand, provide this information. The precision of measurement is currently a constraint of these two technologies due to motion artefacts related to data gathering.

Precision AL measurements are necessary for addition to correct corneal curvature measurements. Optical coherence methods are used in commercially available instruments to determine AL. Three aspects influence accuracy in this case: (1) The signal-to-noise ratio for peak detection; (2) eye motion during an AL scan; and (3) the fluctuation of the ocular media's refractive index. The signal-to-noise ratio and scanning speed are frequently connected. Faster scanning eliminates eye motion abnormalities, but it can potentially lower signals, especially in dense cataracts.

The unknown effective refractive index of the crystalline lens is an unsolved challenge in currently accessible technology. To calculate the geometric AL from the optical coherence signal, the most systems use an ultrasonic equivalent. The accuracy of the AL measurement is harmed since the refractive index varies significantly from eye to eye depending on cataract grade.

ACD and optionally LT have been established as pre-operative characteristics that can aid in the accurate prediction of IOL position. As a result, ocular biometry should be able to reliably and correctly quantify these characteristics.

As used herein, “improper contact lens wear” incudes contact lens overuse, inadequate contact lens cleaning, improper contact lens care, failure to follow contact lens instructions (including frequency of lens replacement, frequency of cleaning, duration of wear), wearing of expired contact lenses, wearing of contact lenses of incorrect prescription (including outdated prescription), and wearing improperly prescribed contact lenses (e.g., poorly fitted contact lenses).

The tear film, with its lipid, aqueous, and mucin layers, normally provides a protective barrier to the corneal tissue and corneal epithelium, and it also has a wetting function to prevent drying or desiccation of the ocular tissues. The tear film also acts as a conduit for the provision of oxygen and nutrients to the corneal epithelial cells, as well as the removal of any potential pathogens, debris and waste products. In many patients preparing for ocular surgery, the tear film may be deficient, unstable or disrupted, for example, as a result of reduced aqueous secretion or increased evaporation of the tear film, or reduced secretion of mucin or lipids. Consequently, the ocular surfaces, including the cornea, may become less protected and vulnerable and/or prone to damage and deterioration.

The term “ocular surface disease (OSD)” relates to diseases or conditions that affect and damage the surface layers of the eyes, such as affecting the cornea, the conjunctiva, and the glandular network (including the Meibomian glands). Ocular surface diseases may include diseases such as dry eye disease (most common subtype of OSD), but also blepharitis, neurotrophic keratitis, ocular rosacea, and Meibomian gland dysfunction. Ocular surface diseases may include any combination of conditions selected from surface damage of the cornea, surface damage of the conjunctiva, tear film deficiency and/or tear film disruption.

The term “corneal surface damage” may include any combination of surface damage of the cornea, including the total corneal region, the central corneal region, the nasal corneal region, the temporal corneal region, and the inferior corneal region. The degree of corneal surface damage may be evaluated by examining the eye using corneal staining techniques, e.g., by staining with fluorescein, which preferentially stains dead and damaged cells of the cornea, thus showing otherwise difficult to observe corneal surface damage.

As used herein, the term “corneal fluorescein staining score” or “corneal staining score” refers to a measurement of corneal staining according to the NEI (National Eye Institute) grading system. In this system, the cornea is divided into five sections (inferior, superior, central, nasal, and temporal regions of the cornea) and assigning a value from 0 (no damage) to 3 (severe damage) to each section, for a maximum of 15 points. The measurement is made by staining the patient's cornea using fluorescein stain. While all 5 corneal sections are taken into account for the total corneal fluorescein staining score (tCFS;0-15), the central corneal staining score(cCFS;0-3) considers only the central corneal region. In some embodiments, a moderate to severely affected patient may have a total corneal fluorescein staining score in excess of 10 or even of 11 at the outset of the treatment period. Because of its importance in vision, the central corneal region is more critical to patient outcomes. In some embodiments, a moderate to severely affected patient may have a central corneal fluorescein staining score in excess of 2 at the outset of the treatment period.

Tear film quality may be evaluated using the Schirmer test type I, and/or the tear film break-up time (TFBUT or TBUT). In some embodiments, prior to the treatment period the patient has an unanesthetized Schirmer's test score in the range of 2 to 8 mm, or 4 to 6 mm.

As used herein, the term “treatment period” or any reference to a length of time of treatment refers to the period of time from the first day to the lasty day that the composition is administered to the eye of the patient according to any of the methods described herein.

In some embodiments according to the present disclosure, the patient may be suffering from dry eye disease. This is a complex disease that results in symptoms of discomfort, visual disturbance, and tear film instability, and which creates potential for damage of the ocular surface. It may be accompanied by increased osmolarity of the tear film and inflammation of the ocular surface. A patient having dry eye disease may experience any one of, or a combination of tear hyperosmolarity, tear film instability or abnormalities in the lipid layer composition of the tear film.

Two major categories of keratoconjunctivitis sicca or dry eye disease (DED) are distinguished today, which are aqueous-deficient DED and evaporative DED. Within the class of aqueous-deficient forms of DED, two major subtypes may be differentiated, Sjögren and non-Sjögren. The Sjögren syndrome can be a primary disease or can result from other autoimmune diseases, such as systemic lupus erythematosus or rheumatoid arthritis. Non-Sjögren patients suffering from an aqueous-deficient DED usually have a lacrimal gland insufficiency, lacrimal duct obstruction or reflex hyposecretion. In the second major class, evaporative dry eye disease, one of the major causes is meibomian gland disease or dysfunction, eyelid aperture disorders, blink disorders (as in Parkinson's disease) or ocular surface disorders (as in allergic conjunctivitis).

The ophthalmic composition according to the present disclosure employs a semifluorinated alkane as a liquid vehicle for the cyclosporine, preferably the compound 1-(perfluorobutyl)pentane. 1-(perfluorobutyl)pentane is a semifluorinated alkane with the chemical formula F(CF₂)₄(CH₂)₅H. It is an inert, water-insoluble liquid, with a density of 1.284 g/cm³ at 25° C. and refractive index of 1.3204 at 20° C. Alternative nomenclature for this compound includes F4H5, wherein F denotes a linear perfluorinated alkane segment comprising 4 carbon atoms and wherein H denotes a linear and non-fluorinated alkane hydrocarbon segment of 5 carbon atoms. Preferably, the 1-(perfluorobutyl)pentane is substantially free of water.

In one embodiment, the ophthalmic composition for any one of the uses or use in any one of the methods according to the present disclosure may comprise or consist, further to the cyclosporine featured in any one the preferred concentrations of the disclosure, of at least about 97% (w/w) or more preferably, of at least about 98% (w/w), or of at least about 98.5% (w/w), or of at least about 99% (w/w) of 1-(perfluorobutyl)pentane, based on the total weight of the ophthalmic composition (final dosage form). In another embodiment, the pharmaceutical composition for any one of the methods or uses according to the present disclosure may consist of, in addition to the cyclosporine in an amount or concentration as defined herein, from about 95.0 to about 99.99% (w/w), or about 98.0 to about 99.99% (w/w), or from about 98.5 to 99.99% (w/w), or from about 99.00 to about 99.9999% (w/w) of 1-(perfluorobutyl)pentane, based on the total weight of the final composition.

In another embodiment, the ophthalmic composition for any one of the uses or use in any one of the methods as described in the present disclosure may optionally further comprise 2-(perfluorobutyl)pentane. The composition, in addition to 1-(perfluorobutyl)pentane, may optionally comprise minor amounts of 2-(perfluorobutyl)pentane, of up to 2% (w/w), or up to 1% (w/w), or up to 0.5% (w/w) or up to 3% (w/w), or up to 0.2% (w/w).

The concentration of cyclosporine in the ophthalmic compositions for any one of the methods or uses according to the disclosure is 0.1% (w/v) of the composition.

Unless otherwise indicated, the term “% (w/v)” denotes the amount of a component of a composition as a weight percentage in relation to the total volume of the composition (with ‘w’ denoting the weight and ‘v’ denoting volume). For example, 0.1% (w/v) would correspond to 1.0 mg of a component in 1 mL of the composition. Unless otherwise indicated, the term “% (w/w)” or wt. % refers to the amount of a component of a composition as a weight percentage in relation to the total weight of the composition (with ‘w’ denoting weight).

The term ‘about’ as used herein and in reference or connection to a parameter, for example such as the concentration of cyclosporine dissolved in the composition or the volume featured in a single dose or applied liquid drop of the composition includes the precise value as defined, as well as any value falling within the degree of variability usually observed in measuring or determining these parameters using the standard techniques and equipment known in the art and field.

A dose of a composition for any one of the methods or uses according to the present disclosure and as described in any one of the embodiments herein is topically administered in the form of a (i.e., one) single drop to an eye of a subject. The drop may be administered to the surface of the eye, preferably to any surface region or tissue of the eye that is accessible to topical administration or instillation, for example to the cornea. The single drop of the composition may be instilled directly onto a surface of the eye, such as the corneal surface of the eye, or alternatively into a space, i.e., sac or pocket, formed by gently pulling down of the lower eyelid of an eye.

As used herein, the term ‘administration to an eye’ or ‘per eye’ refers to the administration of a given dose, e.g., a single dose, of an ophthalmic composition according to the disclosure to an individual eye of a subject.

In an embodiment of the present disclosure, the ophthalmic compositions for any of the methods or uses described herein are administered at a dose of a single drop two times a day per eye. Thus, a patient undergoing treatment for both eyes in accordance with such dosing scheme would receive a total of two drops for each eye on each day of a given treatment period.

Where the ophthalmic composition is administered more than once per day to each eye, for example two times daily per eye, in a further embodiment, the time interval between topical administration of the composition to the eye or eye surface of the first dose and the second dose may be at least 4 hours, or at least 6 hours, or at least 12 hours.

As used herein, the term “up to about” or “up to” used in context of a parameter, such as presently in relation to the amount of ethanol in the composition, refers to any value of the parameter greater than zero and up to, and inclusive of, the defined parameter. For example, an amount of “up to about 1.0% (w/w) of ethanol” should be understood as including any value greater than zero ranging up to and including the value of 1.0% (w/w) of ethanol, and would include, for example, values such as 0.01%, 0.05%, 0.1%, 0.2%, 0.3%, 0.4%, 0.5% 0.6%, 0.7, 0.8, 0.9, 0.95%, 0.99% (w/w) of ethanol, taking into account any degree of variability usually observed in measuring or determining this parameter, using the standard techniques and equipment known in the relevant field.

The absence of an organic co-solvent such as ethanol may offer the advantages of a simpler two component formulation compared to a three-component formulation additionally comprising a co-solvent such as ethanol. The further inclusion, of even one additional composition component may add complexity in terms of factors such as cost, manufacturing, handling, packaging, and also patient compliance.

The compositions for any of the methods or uses of the present disclosure are preferably provided as a clear solution, wherein the cyclosporine is fully dissolved and in solution in the 1-(perfluorobutyl)pentane (at room temperature conditions i.e., between 15 to 25° C.). If ethanol is included, said compositions are also provided as a clear solution of cyclosporine dissolved and in solution in 1-(perfluorobutyl)pentane and the ethanol. Further, if 2-(perfluorobutyl)pentane is included, said compositions are also provided as a clear solution of cyclosporine dissolved in the mixture of 1-(perfluorobutyl)-pentane, 2-(perfluorobutyl)-pentane and ethanol. In one embodiment, the compositions are provided in sterile form.

In another embodiment, the ophthalmic compositions for any one of the methods or uses according to the present disclosure are substantially free of water and/or substantially free of a preservative. As understood herein, the term ‘substantially free’, or alternatively ‘essentially free’ in reference to a composition constituent refers to the presence of said constituent in no more than trace amounts and that if present in trace amounts the constituent provides no technical contribution to the composition.

Preferably, the ophthalmic compositions for any one of the methods or uses according to the present disclosure are substantially free of water, substantially free of a preservative and are effective in inhibiting microbial growth.

In another embodiment, the ophthalmic compositions for any one of the methods or uses according to the present disclosure are characterized by a remarkable wetting and spreading behavior by which they can rapidly and effectively spread over the surface of the eye, such as the corneal surface. Thus, a droplet (drop) of the ophthalmic compositions for any one of the methods or uses according to the present disclosure when administered to the surface of the eye leads to rapid spreading of the compositions over the corneal surface.

Preferably, the ophthalmic compositions for any one of the methods or uses according to the present disclosure form small droplets (drops), in the range of about 8-10 μl, or 9-10 μl, or 9-11 μl, such as about 10 μL or about 9 μl when administered from a drop dispenser.

In another preferred embodiment, the ophthalmic compositions for any one of the methods or uses according to the present disclosure are characterized by the comparable low amount of cyclosporine administered in a single dose per eye, such as about 10 μg cyclosporine administered in a single dose per eye, or about 8 to 10 μg or 9 to 10 μg of cyclosporin administered in a single dose per eye.

As used herein, the term “consists” and related terms “consisting” or “consist” is to be understood as meaning that no other features, other than those prefaced by the term are present. In the context of the ophthalmic compositions as described herein, if any other constituent or component is present in the composition other than those prefaced by such term, then it is present only in trace or residual amounts such as to confer no technical advantage or relevance in respect of the object of the disclosure, such as may be further understood by the term ‘essentially” or “substantially” used in conjunction with these terms (e.g., ‘essentially consisting of”).

The methods and compositions for use described herein are preferably targeted towards human subjects or patients.

As described above, the present disclosure contemplates a kit comprising an ophthalmic composition as described herein with a container for holding the ophthalmic composition and a drop dispenser adapted for administering about 8 to 10 μL, or about 9 to 11 μl, or about 9 to 10 μl, or about 10 μl or about volume of the composition per drop. The kit may also further comprise instructions for use, for example in the form of a leaflet, packaging or other readable means, indicating use in accordance with any one of the uses or methods described herein.

As understood herein, the drop dispenser may be a dispenser or applicator means which may be mounted, fixed or connected to the container for holding the ophthalmic composition. Preferably, the drop dispenser is adapted for dispensing a single dose in the form of a single drop of the composition. More preferably, the drop dispenser is adapted for dispensing a single dose of about 8- to 10-μl volume, or about 9- to 10-μl volume or is adapted for dispensing a single dose of about 10-μl volume, or of about 9 μl volume.

The container for holding the ophthalmic composition as understood herein is preferably of a volume which may hold a single dose, but more preferably of a volume which may hold multiple or a plurality of doses of the composition.

The following examples serve to illustrate the invention, however should not be understood as restricting the scope of the invention.

### EXAMPLES

The investigational drug product CyclASol® is a clear, ophthalmic solution of 0.1% (w/v) cyclosporine A dissolved in 1-perfluorobutylpentane (F4H5) with 1.0% (w/w) ethanol as co-solvent. It has been investigated for use as a treatment for dry eye disease in several international clinical trials, including Phase I, Phase II and Phase III trials, with very promising results for dry eye disease treatment.

### Example 1

In the recently completed ESSENCE 2 trial, the efficacy, safety and tolerability of CyclASol® 0.1% ophthalmic solution was studied for the treatment of dry eye disease. The study was performed as a randomized, double-blind trial. 834 participants were enrolled, with 423 assigned to the treatment group and 411 assigned to the placebo group. The treatment group received 29 days of daily treatment with CyclASol 0.1% twice per day, which the placebo group received the vehicle (F4H5 with 0.1% (w/w) ethanol).

At the conclusion of the initial 29-day period of the study, the primary endpoints were analyzed: total corneal fluorescein staining score (tCFS, NEI scale) and dryness score (Visual Analog Scale, VAS). In addition, the secondary endpoints total conjunctival lissamine green staining score (tCLGS, Oxford scale), central corneal fluorescein staining score (cCFS, NEI scale), and blurred vision score (VAS).

Key inclusion criteria were that the patients had at least 6 months of dry eye disease prior to enrollment, with a dryness score on the VAS of at least 50, a tCFS score of at least 10(NEI scale), a tCLGS of at least 2 (Oxford scale), and an unanesthetized Schirmer's test score of 1 to 10 mm. Enrolled patient characteristics were as follows:

| Characteristic | Treatment Group | Placebo Group |
|---|---|---|
| Mean age | 58 | 57 |
| Mean tCFS at baseline | 11.5 | 11.5 |
| Mean cCFS at baseline | 2.1 | 2.1 |
| Mean tCLGS at baseline | 3.7 | 3.8 |
| VAS: dryness score at baseline | 70.4 | 70.0 |
| VAS: Blurred vision score at baseline | 53.4 | 51.9 |
| Mean Schirmer score at baseline | 5.1 mm | 4.8 mm |

At the 29-day evaluation, the patient's characteristics improved as follows:

| | Treatment | Placebo | |
|---|---|---|---|
| Characteristic | Group | Group | p-value |
| Change in tCFS at day 29 | −3.0 | −3.6 | 0.03 |
| Change in cCFS at day 29 | −0.8 | −0.7 | 0.05 |
| Change in tCLGS at day 29 | −1.2 | −0.9 | 0.0009 |
| Participants with ≥3 grade | 72% | 60% | 0.0002 |
| tCFS improvement at day 29 | | | |
| Participants with ≥1 grade | 67% | 60% | 0.04 |
| cCFS improvement at day 29 | | | |
| Eyes with ≥10 mm Schirmer | 11% | 7% | 0.05 |
| improvement at day 29 | | | |

These results demonstrate statistically significant improvement in the primary and secondary endpoints in the chart above after 29 days of treatment with CyclASol® 0.1% ophthalmic solution. Importantly, at 29 days, the degree of corneal surface damage was effectively decreased, as evidenced by a decrease of >3 grades in tCFS in 72% of participants and by a decrease of >1 grades in cCFS in 67% of participants.

Following the initial 29-day phase of the study, a subset of 177 of the total 202 patients were followed for a total of 12-months in an open-label safety extension (all patients rolled over to a single treatment group). It was unexpectedly found that at 6 months, both tCFS and Schirmer results continued to improve steadily in the treatment group. For example, tCFS fell to 39% below baseline in the treatment group at day 39, and further fell to 53% below baseline at 6 months. Similarly, the Schirmer test result increased by 45% from baseline at day 29, and further increased to 70% improvement from baseline at 6 months.

In addition, the study results demonstrated comparable safety profile between treatment and placebo groups. Importantly, less than 10% of treatment group patients reported mild instillation site reactions, only 0.2% of treatment group patients reported moderate instillation site reaction (1 patient), and no patients reported severe instillation site reaction. These results are comparable to the placebo group, and demonstrate that, unlike Restasis, CyclASol is much more tolerable for patients, thus resulting in improved patient compliance.

### Example 2

A compromised ocular surface may impact biometry and refractive outcomes after cataract surgery leading to patient dissatisfaction. Analysis of pooled data from the ESSENCE-1 and ESSENCE-2 clinical trials evaluated the coexistence of cataract and dry eye disease (DED) and the efficacy of a water-free CyclASol eye drops (0.1% (w/v) cyclosporine dissolved in a vehicle comprising 1-perflurobutylpentane and 1 wt. % ethanol) in DED patients with concomitant cataract.

The analyses were performed using data from two prospective, randomized, double-masked, controlled phase III DED trials, ESSENCE-1 and ESSENCE-2 (NCT03292809/NCT04523129). Both studies demonstrated significant improvements of the ocular surface (e.g., at 2-weeks timepoint).

Pooled data were stratified into 3 groups: patients without cataract, patients with concomitant cataract, and pseudophakic patients. Key outcome measures were change from baseline in total corneal fluorescein staining (tCFS) (0-15) and central corneal fluorescein staining (cCFS) at Day 15 and Day 29. Responder analyses were carried out for tCFS (≥3 grades improvement) and cCFS (≥1grade improvement) as well.

The analyses included 1162 patients with moderate to severe DED; 35% had coexisting cataract, 20% were pseudophakic and 45% had no cataract. Demographics or baseline DED characteristics were comparable across groups except for age and vision.

Pseudophakic patients and patients with cataract had worse vision and were older compared to patients without cataracts.

The CyclASol eye drops showed a statistically significant reduction compared to its vehicle in the tCFS score at Day 15 and Day 29 in all three subgroups. In the subgroups with cataract, 62% of patients treated with CyclASol eye drops were tCFS responders and 63% of patients were cCFS responders Thus, the degree of corneal surface damage, being an important indicator of ocular surface disease, effectively improved in the cataract subgroup. The results for the cataract subgroup at Day 29 are shown in the following table:

Subgroup: Patients with Cataracts (Day 29)

| | Treatment | Placebo | |
|---|---|---|---|
| | Group | Group | |
| Characteristic | (n = 210) | (n = 189) | p-value |
| Participants with ≥3 grade tCFS | 62% | 45% | 0.0062 |
| improvement (tCFS responders) | | | |
| Participants with ≥1 grade cCFS | 63% | 47% | 0.0059 |
| improvement (cCFS responders) | | | |

Further, in the subgroups with cataract, 56% of patients treated with CyclASol eye drops were tCFS responders and 59% of patients were cCFS responders at Day 15. Thus, the degree of corneal surface damage, being an important indicator of ocular surface disease, effectively improved in the cataract subgroup in only 15 days of treatment. The results for the cataract subgroup at Day 15 are shown in the following table:

Subgroup: Patients with Cataracts (Day 15)

| | Treatment Group | Placebo Group |
|---|---|---|
| Characteristic | (n = 210) | (n = 189) |
| Participants with ≥3 grade tCFS | 56% | 43% |
| improvement (tCFS responders) | | |
| Participants with ≥1 grade cCFS | 59% | 43% |
| improvement (cCFS responders) | | |

The CyclASol eye drops demonstrated rapid and clinically meaningful improvements in corneal staining in patients with DED and cataract comparable to the overall population. The magnitude of the effect and onset of action and makes this treatment an ideal candidate for pre-operative ocular surface disease management of cataract patients.

1. A composition comprising about 0.1% (w/v) cyclosporine dissolved in 1-perfluorobutylpentane, optionally with about 1% (w/w) ethanol, for use in a method of preparing a patient for ocular surgery (e.g., cataract surgery), the method comprising topically administering the composition two times daily to the eye of the patient (e.g., the eye undergoing ocular surgery).

2. A composition comprising about 0.1% (w/v) cyclosporine dissolved in 1-perfluorobutylpentane, optionally with about 1% (w/w) ethanol, for use in a method of treating corneal surface damage in a patient preparing for ocular surgery (e.g., cataract surgery), the method comprising topically administering the composition two times daily to the eye of the patient.

3. The composition for use according to any one of claims 1-2, wherein the method further comprises the step of performing biometry measurements in preparation for the ocular surgery.

4. A composition comprising about 0.1% (w/v) cyclosporine dissolved in 1-perfluorobutylpentane, optionally with about 1% (w/w) ethanol for use in a method of performing biometry measurements for ocular surgery (e.g., cataract surgery), the method comprising topically administering the composition two times daily to the eye of the patient, prior to the biometry measurements.

5. The composition for use according to any one of claims 1-4, wherein the composition comprises about 0.1% (w/v) cyclosporine dissolved in 1-perfluorobutylpentane with about 1% (w/w) ethanol, and optionally up to about 1% (w/w) 2-perfluorobutylpentane.

6. The composition for use according to any one of claims 1-5, wherein the composition consists of about 0.1% (w/v) cyclosporine dissolved in 1-perfluorobutylpentane with about 1% (w/w) ethanol, and optionally up to about 0.5% (w/w) 2-perfluorobutylpentane.

7. The composition for use according to any one of claims 1-6, wherein the amount of cyclosporine administered in a single dose per eye is about 8-12 μg, e.g., 9-11 μg, or about 10 μg cyclosporine.

8. The composition for use according to any one of claims 1-7, wherein the patient is administered the composition for at least a period of 7 days, or for at least 14 days, or for at least 21 days, or for at least 28 days or for at least 35 days or for at least 42 days.

9. The composition for use according to any of the claims 1-8, wherein the method further comprises the step of determining the degree of corneal surface damage in preparation for the ocular surgery.

10. The composition for use according to claim 9, wherein the degree of corneal surface damage is determined by the total corneal fluorescein staining score and/or the central corneal fluorescein staining score.

11. The composition for use according to any one of claims 1-10, wherein, before the beginning of the administration of the composition according to the method, the patient is characterized by the eye intended for ocular surgery having:
(a) a total corneal fluorescein staining score of greater than or equal to 7 (e.g., greater than 8, 9, 10, 11, or 12), such as 7-15, 7-13, 10-15 or 10-13;
(b) a central corneal fluorescein staining score of equal or greater than 1 (e.g., greater than 1, greater than 2), such as 1-2 or 1-3 or 2-3.

12. The composition for use according to any one of claims 1-11, wherein after the period of treatment with the composition (e.g., after administration of the composition for at least a period of 7 days, or for at least 14 days, or for at least 21 days, or for at least 28 days or for at least 35 days or for at least 42 days) according to the method, the patient is characterized by the eye intended for ocular surgery having:
(a) a total corneal fluorescein staining score that has decreased by at least 3 grades; and/or
(b) a central corneal fluorescein staining score that has decreased by at least 1 grade.

13. The composition for use according to any one of claims 1-12, wherein the method improves the patient's total corneal fluorescein staining score in the eye intended for ocular surgery by at least 3 grades before the biometry measurements are performed and/or before the ocular surgery is performed.

14. The composition for use according to any one of claims 1-13, wherein the method improves the patient's central corneal fluorescein staining score in the eye intended for ocular surgery by at least 1 grade before the biometry measurements are performed and/or before the ocular surgery is performed.

15. The composition for use according to any one of claims 1 to 14, wherein the step of determining the degree of corneal surface damage precedes the administration of the composition.

16. The composition for use according to any one of claims 1 to 15, wherein the patient is subjected to biometry not before a reduction of the patient's total corneal fluorescein staining score in the eye intended for ocular surgery by at least 3 grades is achieved.

17. The composition for use according to any one of claims 1 to 16, wherein the composition is administered to the patient until a reduction of the patient's total corneal fluorescein staining score in the eye intended for ocular surgery by at least 3 grades is achieved.

18. The composition for use according to any one of claims 1 to 17, wherein the patient suffers from corneal surface damage not caused by dry eye disease.

19. The composition for use according to any one of claims 1 to 18, wherein the administration of the composition continues after the ocular surgery.
